(19) United States
(12) Patent Application Publication
(10) Pub. No.: US 2026/0348976 A1
(43) Pub. Date: Nov. 5, 2026

(54) DIAGNOSTIC AGENT

(71) Applicant: Citryll B.V., Oss (NL)

(72) Inventors: Helmuth Hendrikus Gerardus VAN ES, Oss (NL); Renato Gerardus Silvano CHIRIVI, Oss (NL); Annemarie KIP, Oss (NL)

(73) Assignee: Citryll B.V., Oss (NL)

(21) Appl. No.: 19/698,797

(22) Filed: Jun. 4, 2026

Related U.S. Application Data
(63) Continuation of application No. PCT/EP2024/085065, filed on Dec. 6, 2024.

(30) Foreign Application Priority Data
Dec. 7, 2023 (GB) 2318703.2

(51) Int. Cl. G01N 33/68; G01N 33/58

(52) U.S. Cl. CPC G01N 33/6875; G01N 33/58; G01N 2440/18; G01N 2800/102

(57) ABSTRACT

The present invention relates to antibodies or binding fragments thereof for use in methods of identifying or diagnosing diseases associated with extracellular trap formation or release from cells, such as Neutrophil Extracellular Trap (NET)-associated pathologies or Eosinophil Extracellular Trap (EET)-associated pathologies.

### CROSS-REFERENCE TO RELATED APPLICATIONS

This application is a continuation of International Application No. PCT/EP2024/085065, entitled “DIAGNOSTIC AGENT,” filed internationally on Dec. 6, 2024, which claims priority to GB provisional application No. 2318703.2, entitled “DIAGNOSTIC AGENT,” filed on Dec. 7, 2023, the contents of each of which are incorporated by reference in its entirety.

### INCORPORATE BY REFERENCE OF SEQUENCE LISTING

The present application is being filed along with a Sequence Listing in electronic format. The Sequence Listing is provided as a file entitled 251502015101SeqList.XML, created Jun. 4, 2026, which is 57,246 bytes. The information in the electronic format of the Sequence Listing is herein incorporated by reference in its entirety.

### FIELD OF THE INVENTION

The present invention relates to antibodies or binding fragments thereof for use in methods of identifying or diagnosing diseases associated with extracellular trap release from cells, such as Neutrophil Extracellular Trap (NET)-associated pathologies or Eosinophil Extracellular Trap (EET)-associated pathologies.

### BACKGROUND OF THE INVENTION

Whilst the inflammatory process plays a key role in the normal response of the body to infection, in inflammatory disorders the inflammatory process forms part of the underlying pathogenesis of the disorder, for example because the immune system is triggered against the body's own tissues or because the nature of the immune response against a pathogen is too great and causes damage to the body. Given the large number of disorders inflammation plays a role in, it represents a substantial problem in the healthcare industry. Inflammation is often subdivided into acute and chronic inflammation. Chronic inflammation is considered to be inflammation of a prolonged duration (weeks or months) in which active inflammation, tissue destruction and attempts at healing are proceeding simultaneously. Although chronic inflammation can follow an acute inflammatory episode, it can also begin as an insidious process that progresses with time, for example, as a result of a persistent infection (e.g., tuberculosis, syphilis, fungal infection) that causes a delayed hypersensitivity reaction, prolonged exposure to endogenous (e.g., elevated plasma lipids) or exogenous (e.g., silica, asbestos, cigarette tar, surgical sutures) toxins, or autoimmune reactions against the body's own tissues (e.g., rheumatoid arthritis, systemic lupus erythematosus, vasculitis, multiple sclerosis, psoriasis).

Inflammatory disorders include those where the focus of the research has perhaps been on other aspects of the disorder. For example, one such condition is Parkinson's disorder (PD) where much research has focused on the formation of neurofibrillary tangles of tau protein. The underlying pathogenesis of PD though also involves inflammation which is thought responsible for the underlying damage to the central nervous system (CNS), with neutrophils considered to play a role in that inflammation.

One consequence of inflammation is the formation of Neutrophil Extracellular Traps (NETs). NETs are also known to cause inflammation. NETs are structures comprising DNA and histones that are produced by neutrophils as part of the host defense mechanism against pathogens. They can trap and kill various bacterial, fungal, viral and protozoal pathogens, and their release is one of the first lines of defense against pathogens. Following activation by microorganisms or cytokines, histones become hypercitrullinated and the neutrophil nucleus undergoes a process of chromatin decondensation that leads to the formation of NETs by NETosis, a form of neutrophil cell death.

NETs play a pathological role in a variety of diseases, for example by causing aberrant inflammation. Thus, NETs are involved in the pathology of a variety of inflammatory conditions, such as systemic lupus erythematosus (SLE), lupus, sepsis, vasculitis, inflammatory arthritis, rheumatoid arthritis and osteoarthritis, psoriasis, dermatomyositis, polymyositis, inclusion body myositis, hidradenitis suppurativa, Alzheimer's disease, autoimmune hepatitis, juvenile idiopathic arthritis, myositis (polymyositis and dermatomyositis), Sjögren's disease, Bechet's disease, spondylitis, spondyloarthropathy, multiple system atrophy, Parkinson's disease, Lewy body dementia asthma, allergic rhinovirus exacerbated asthma, COPD, Acute respiratory distress syndrome, cystic fibrosis, idiopathic pulmonary fibrosis, heart failure and atherosclerosis. For example, NETs can cause autoantigen exposure to the extracellular space and the subsequent production of pathological autoantibodies by the subject. Furthermore, NETs and NET remnants harbor toxic histones, which induce vascular damage and subsequent organ damage and failure.

Thus, in such diseases, interfering with NET formation, and inducing clearance of NETs and NET remnants from circulation and tissues, would have therapeutic benefits. Neutrophils are also increasingly being recognized as an important element in tumour progression. They have been shown to exert important effects at nearly every stage of tumour progression with a number of studies demonstrating that their presence is critical to tumour development. Studies have also implicated NETs as facilitators of tumour progression and metastasis. It has also been shown that neutrophils, through the generation of NETs, provide a scaffold and a stimulus for platelet adhesion, thrombus formation and coagulation in tumours.

NETs have also been implicated in reducing organ health after transplant. NETs contribute to primary graft dysfunction, contributing to early mortality after lung transplantation. It has been shown that NETs play a pathogenic role in solid organ transplantation.

A promising approach for the treatment of inflammatory diseases by targeting the production of NETs is the use of antibodies that bind to citrullinated epitopes on the amino terminus of histones 2A and/or histone 4. These antibodies can be used to treat diseases or pathologies associated with citrullination, such as NET-associated pathologies and inflammatory conditions. Antibodies that bind to citrullinated epitopes on deiminated human histone 2A and histone 4 are described in WO2009147201, WO2011070172, WO2016092082 and in particular in WO 2020/038963.

Eosinophils are a form of circulating leukocyte, typically representing about 1 to 3% of white blood cells (WBCs) in a healthy human. They have a wide variety of roles in homeostasis and various diseases including allergy and infection. It has been identified that a particular mechanism of active cytolytic eosinophil cell death releases eosinophil extracellular traps (EETs) and total cellular contents. This is referred to as eosinophil extracellular trap cell death (EETosis). It has also been shown that Charcot-Leyden crystals (a classical pathological marker of eosinophilic inflammation) is associated with EETosis, and the presence of EETosis and EETs has been reported in multiple diseases.

Antibodies that bind to citrullinated epitopes on the amino terminus of histones 2A and/or histone 4 and are able to inhibit EETosis are disclosed in WO2022233931. WO2022233931 discloses methods for the treatment or prevention of an EET-associated pathology using said antibodies. Such pathologies may include: an eosinophilic disease of the skin; a respiratory eosinophilic disease; a gastro-intestinal eosinophilic disease; an allergic disease; or a helminth, fungal, viral, or bacterial infection. In addition, arteriosclerosis or vasculitis may also be treated or prevented.

However, there remains a need for improved methods of identification or diagnosis of the diseases described above.

### SUMMARY OF THE INVENTION

The present inventors have identified that antibodies or binding fragments that specifically bind to a citrullinated epitope on deiminated human histone 2A and/or histone 4 have particular advantages for identifying or diagnosing diseases associated with extracellular trap release from cells, such as Neutrophil Extracellular Trap (NET)-associated pathologies or Eosinophil Extracellular Trap (EET)-associated pathologies.

Accordingly, the present invention provides a method of identifying or diagnosing a human subject with a pathology associated with extracellular trap formation or release from cells comprising the use of an antibody or antigen binding fragment thereof that specifically binds to a citrullinated epitope on deiminated human histone 2A and/or histone 4 as a diagnostic agent.

In a further embodiment, the invention provides a method of identifying or diagnosing a human subject with a pathology associated with extracellular trap formation or release from cells comprising administering a labelled antibody or antigen binding fragment thereof that specifically binds to a citrullinated epitope on deiminated human histone 2A and/or histone 4 to said subject, and diagnosing said pathology based on the localisation of the labelled antibody or antigen binding fragment in the human subject.

In a further embodiment, the invention provides a method of identifying or diagnosing a human subject with a pathology associated with extracellular trap formation or release from cells comprising acquiring an image of the human subject to whom an amount of a labelled antibody or antigen binding fragment thereof that specifically binds to a citrullinated epitope on deiminated human histone 2A and/or histone 4 has been administered, wherein diagnosing said pathology is based on the localisation of the labelled antibody or antigen binding fragment in the image.

Representative antibodies that bind to citrullinated epitopes on deiminated human histone 2A and histone 4 are described in, for example, WO 2020/038963 and WO 2022/233931. Both documents, and the antibodies disclosed therein (including all CDR sequences, variable region sequences and constant region sequences of both the heavy and light chains), are herein incorporated by reference. The antibodies disclosed with identifiers of the format hMQ22.101x/y in WO2020038963, and any antigen binding fragment thereof, are each incorporated by reference. The antibody referred to in WO2020038963 as hMQ22.101f/LC41 is most preferred. This antibody may be described herein as CIT-013.

### BRIEF DESCRIPTION OF THE SEQUENCE LISTING AND NOMENCLATURE

### Antibody Nomenclature

CDR=complementarity-determining region.
VH=heavy chain variable domain.
VL=light chain variable domain.
CH=heavy chain constant domain.
CL=light chain constant domain.
msVH22.101=mouse VH of therapeutic antibody.
msVL22.101=mouse VL of therapeutic antibody.
hVH22.101x=humanized VH of therapeutic antibody, ‘x’ refers to the heavy chain.
hVL22.101y=humanized VL of therapeutic antibody, ‘y’ refers to the light chain.
hVH22.101(HC)x=optimized humanized VH of therapeutic antibody, ‘(HC)x’ refers to the heavy chain.
hVL22.101(LC)y=optimized humanized VL of therapeutic antibody, ‘(LC)y’ refers to the light chain.
hMQ22.101x/y=humanized therapeutic antibody, ‘x’ refers to the heavy chain, ‘y’ refers to the light chain.
hMQ22.101(HC)x/(LC)y=optimized humanized therapeutic antibody of the invention, ‘(HC)x’ refers to the heavy chain, ‘(LC)y’ refers to the light chain.

| SEQ ID | | |
|---|---|---|
| NO | Protein | Name |
| 1 | protein | CDR1 of msVH22.101 and hVH22.101(HC)x |
| 2 | protein | CDR2 of msVH22.101 and hVH22.101(HC)x |
| 3 | protein | CDR3 of msVH22.101 and hVH22.101(HC)x |
| 4 | protein | CDR2 of msVL22.101 and hVL22.101(LC)y |
| 5 | protein | CDR3 of msVL22.101 and hVL22.101(LC)y |
| 6 | protein | CDR1 of hVL22.101LC17 |
| 7 | protein | CDR1 of hVL22.101LC21 |
| 8 | protein | CDR1 of hVL22.101LC27 |
| 9 | protein | CDR1 of hVL22.101LC41 |
| 10 | protein | CDR1 of hVL22.101LC42 |
| 11 | protein | hVH22.101f |
| 12 | protein | hVH22.101HC9 |
| 13 | protein | hVL22.101LC17 |
| 14 | protein | hVL22.101LC21 |
| 15 | protein | hVL22.101LC27 |
| 16 | protein | hVL22.101LC41 |
| 17 | protein | hVL22.101LC42 |
| 18 | protein | SEQ ID NO 1 from WO2016092082-Example 1, |
| | | histone 2A |
| 19 | protein | SEQ ID NO 2 from WO2016092082, histone 4 |
| 20 | protein | Shortened SEQ ID NO 2 from WO2016092082- |
| | | Example 7, histone 4 |
| 21 | protein | Peptide no 4 (human histone 2A) (SEQ ID NO |
| | | 24 from WO2011070172) |
| 22 | protein | Peptide no 6 (human histone 2A) (SEQ ID NO |
| | | 26 from WO2011070172) |
| 23 | protein | Human heavy chain constant domain of IgG1 |
| 24 | protein | Human kappa chain constant domain |
| 25 | protein | msVH22.101 |
| 26 | protein | hVH22.101j |
| 27 | protein | hVH22.101HC7 |
| 28 | protein | hVH22.101HC8 |
| 29 | protein | hVH22.101HC10 |
| 30 | protein | msVL22.101 |
| 31 | protein | hVL22.101e |
| 32 | protein | hVL22.101g |
| 33 | protein | hVL22.101h |
| 34 | protein | hVL22.101i |
| 35 | protein | hVL22.101j |
| 36 | protein | CDR1 of msVL22.101 and hVL22.101g |
| 37 | protein | CDR1 of hVL22.101e |
| 38 | protein | CDR1 of hVL22.101h |
| 39 | protein | CDR1 of hVL22.101i |
| 40 | protein | CDR1 of hVL22.101j |
| 41 | protein | CDR1 of hVL22.101LC16 |
| 42 | protein | CDR1 of hVL22.101LC19 |
| 43 | protein | CDR1 of hVL22.101LC20 |
| 44 | protein | CDR1 of hVL22.101LC22 |
| 45 | protein | CDR1 of hVL22.101LC23 |
| 46 | protein | CDR1 of hVL22.101LC24 |
| 47 | protein | CDR1 of hVL22.101LC25 |
| 48 | protein | CDR1 of hVL22.101LC26 |
| 49 | protein | CDR1 of hVL22.101LC37 |
| 50 | protein | CDR1 of hVL22.101LC38 |
| 51 | protein | CDR1 of hVL22.101LC39 |
| 52 | protein | CDR1 of hVL22.101LC40 |
| 53 | protein | msFibβ XG (SEQ ID NO 37 from WO2011070172) |
| 54 | protein | msVim XS/XL (SEQ ID NO 38 from |
| | | WO2011070172) |
| 55 | Protein | Region around CDR2 of msVL22.101 and |
| | | hVL22.101(LC)y |
| 56 | Protein | Heavy chain constant domain of hCH22.101f |

### BRIEF DESCRIPTION OF THE FIGURES

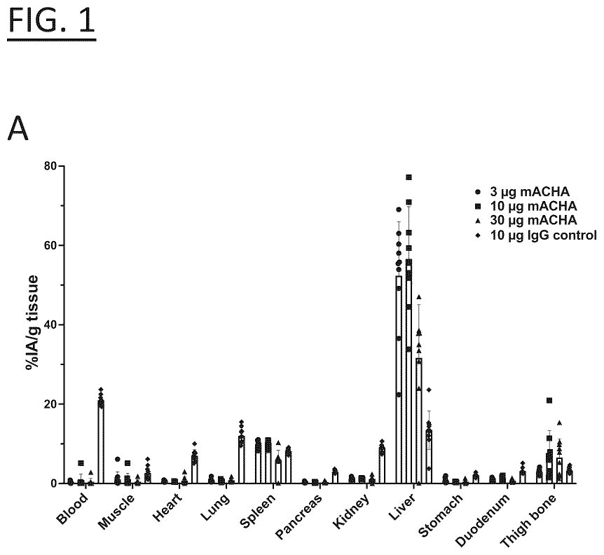

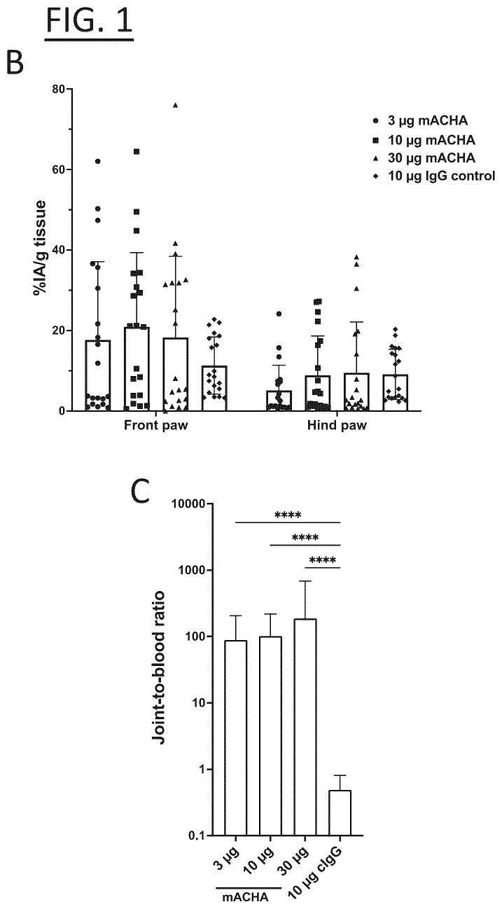

FIG. 1: Uptake of [¹¹¹In]In-mACHA in tissues of mice with collagen induced arthritis
(A) Uptake of radiolabeled antibodies in dissected mouse organs (n=10 per group). Biodistribution was performed 24 hours after injection of mice with 3, 10 or 30 μg [¹¹¹In]Jn-DTPA-mACHA or 10 μg [¹¹¹In]In-DTPA-cIgG. mACHA clears faster from the circulation compared to cIgG.
(B) Uptake of radiolabeled antibodies in the front and hind paws (n=10 per group).
(C) Antibody joint-to-blood ratio, average from front and hind paws, shows ±100-fold increase of [¹¹¹In]In-DTPA-mACHA uptake in paws. The higher [¹¹¹In]In-DTPA-cIgG blood level results in a blood-to-joint ratio of <1, indicating a specific uptake (n=10 per group resulting in 40 paws per group). Joint-to-blood ratio is significantly higher in the mACHA dosed groups compared to cIgG (****=p<0.0001), but not between mACHA dosed groups (Kruskal-Wallis test, Dunn's multiple comparisons).
% IA/g tissue=% injected activity per gram tissue.

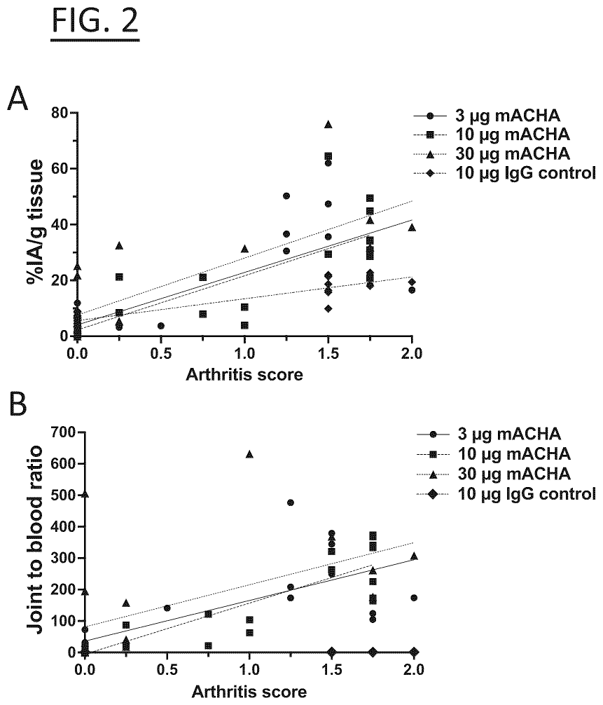

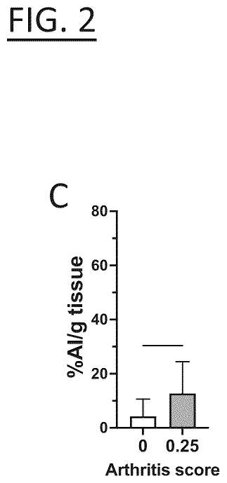

FIG. 2: Uptake of [¹¹¹In]In-DTPA-mACHA correlates with arthritic score
(A) Correlation of antibody uptake with increasing arthritic score is visualized for the front paws, for the four different dose groups, by using simple linear regression. Correlations were determined using non-parametric Spearman correlation. r equals 0.77, 0.84 and 0.80 for the 3, 10 and 30 μg dose groups, respectively. p-values were all <0.0001. For the 10 μg cIgG group r=0.86 (p<0.0001), due to enhanced permeability and retention (EPR) in the inflamed joints.
(B) Correlating joint-to-blood ratio with increasing arthritic score corrects the % IA/g of uptake in the joints for levels circulating in blood. Simple linear regression was used to visualize the relationship. Correlations were determined using non-parametric Spearman correlation. r equals 0.77, 0.90 (both p<0.0001) and 0.67 (p=0.0013) for the 3, 10 and 30 μg dose groups, respectively. For the 10 μg cIgG group r=0.85 (p<0.0001) due to EPR in the inflamed joints. As the correlation coefficient was highest for 10 μg [¹¹¹In]In-DTPA-mACHA for both (A) and (B), this was chosen as the optimal dose.
(C) Uptake of [¹¹¹In]In-DTPA-mACHA was significantly increased (p=0.005) in mild arthritic paws (arthritic score=0.25, n=6) compared to unaffected paws (arthritic score=0, n=25), as calculated by Mann-Whitney test. Paws of all 3 dose groups were included in this calculation.
% IA/g tissue=% injected activity per gram tissue.

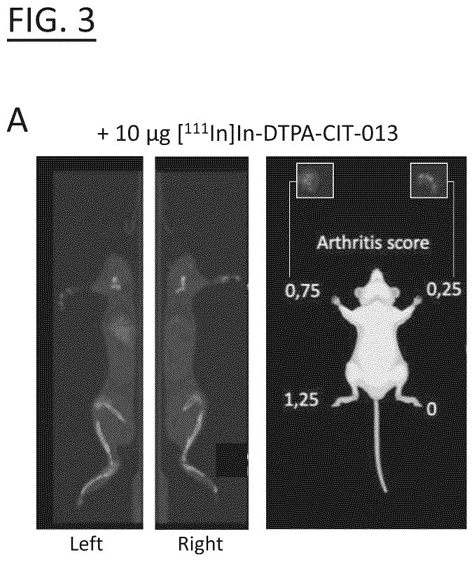

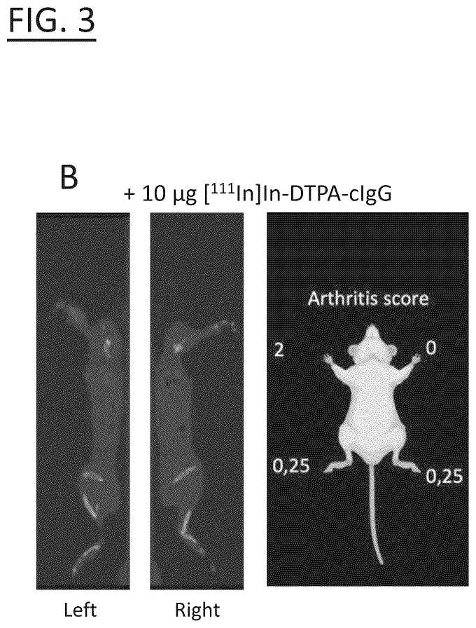

FIG. 3: SPECT/CT images showing antibody homing in arthritic joints.
(A) Exemplary sagittal planes of SPECT/CT imaging showing the specific localization of [¹¹¹In]In-DTPA-mACHA (or [¹¹¹In]In-DTPA-CIT-013) into the ankle and toes of the left hind paw, matching with the arthritic score of 1.25. Additional uptake can be seen in the left and right knee and left shoulder. These joints are not part of the scoring system as macroscopical swelling cannot be observed in those areas. The two insets above the mouse schematic image are different coronal planes showing the uptake in the front paws, as this could not be made visible in the same sagittal planes as the hind paws.
(B) Exemplary sagittal planes of SPECT/CT imaging showing the distribution of [¹¹¹In]In-DTPA-cIgG. Uptake of control IgG is seen in the severely affected paw, but showing a distinctive pattern compared to mACHA. This diffuse pattern around the paw is consistent with the enhanced permeability and retention of antibodies in inflamed tissue.

In both treatment groups, signal is present in the liver which is due to antibody clearance by the liver.

### DETAILED DESCRIPTION OF THE INVENTION

It is to be understood that different applications of the disclosed invention may be tailored to the specific needs in the art. It is also to be understood that the terminology used herein is for the purpose of describing particular embodiments of the invention only and is not intended to be limiting.

In addition, as used in this specification and the appended claims, the singular forms “a”, “an”, and “the” include plural references unless the content clearly dictates otherwise. Thus, for example, reference to “an antibody” includes “antibodies”, and the like.

Where the term “comprising” is used herein, also provided is an embodiment “consisting essentially of” or “consisting of” what is set out.

The term “about” in relation to a point value indicated herein in one embodiment means that what is provided is within ±10% of the stated point value. In one preferred embodiment, it is within ±5%. In one more preferred embodiment, it is within ±1%. When used in relation to a numerical range, in one embodiment the amount that each point value used to indicate the endpoint of a range me value is within any of those values.

All publications, patents and patent applications cited herein, whether supra or infra, are hereby incorporated by reference in their entirety.

### Methods of Diagnosis

The present invention is concerned with the identification of an epitope in a human subject with a pathology associated with extracellular trap formation or release from cells, wherein the epitope is a citrullinated epitope on deiminated human histone 2A and/or histone 4. The presence and localisation of said epitope in said human subject indicates that said human subject has a pathology associated with extracellular trap formation or release from cells. In a preferred embodiment, the pathology associated with extracellular trap formation or release from cells is a Macrophage Extracellular Trap (MET)-associated pathology, Mast Cell Extracellular Trap (MCET)-associated pathology or Monocyte derived Extracellular Trap-associated pathology. In a preferred embodiment, the pathology associated with extracellular trap formation or release from cells is a NET-associated pathology or an EET-associated pathology. The presence and localisation of said epitope in said human subject therefore indicates that said human subject has a NET-associated pathology or an EET-associated pathology. In a preferred embodiment of the invention the NET-associated pathology is diagnosed at an early stage. In a preferred embodiment of the invention the EET-associated pathology is diagnosed at an early stage.

Therefore, the present invention is concerned with a method of identifying or diagnosing a human subject with a pathology associated with extracellular trap formation or release from cells comprising the use of an antibody or antigen binding fragment thereof that specifically binds to a citrullinated epitope on deiminated human histone 2A and/or histone 4 as a diagnostic agent. The present invention is also concerned with a method of identifying or diagnosing a pathology associated with extracellular trap formation or release from cells in a human subject comprising the use of an antibody or antigen binding fragment thereof that specifically binds to a citrullinated epitope on deiminated human histone 2A and/or histone 4 as a diagnostic agent.

Therefore, the present invention is concerned with a method of identifying or diagnosing a human subject with a NET-associated pathology comprising the use of an antibody or antigen binding fragment thereof that specifically binds to a citrullinated epitope on deiminated human histone 2A and/or histone 4 as a diagnostic agent. The present invention is also concerned with a method of identifying or diagnosing a NET-associated pathology in a human subject comprising the use of an antibody or antigen binding fragment thereof that specifically binds to a citrullinated epitope on deiminated human histone 2A and/or histone 4 as a diagnostic agent.

In a preferred embodiment of the invention, the antibody or antigen binding fragment thereof is conjugated to an additional moiety. In a preferred embodiment, the antibody or antigen binding fragment thereof is labelled. In a preferred embodiment, the label is a radioactive label.

The present invention is also concerned with method of identifying or diagnosing a human subject with an EET-associated pathology comprising the use of an antibody or antigen binding fragment thereof that specifically binds to a citrullinated epitope on deiminated human histone 2A and/or histone 4 as a diagnostic agent. The present invention is also concerned with a method of identifying or diagnosing a an EET-associated pathology in a human subject comprising the use of an antibody or antigen binding fragment thereof that specifically binds to a citrullinated epitope on deiminated human histone 2A and/or histone 4 as a diagnostic agent.

In a preferred embodiment of the invention, the antibody or antigen binding fragment thereof is conjugated to an additional moiety. In a preferred embodiment, the antibody or antigen binding fragment thereof is labelled. In a preferred embodiment, the label is a radioactive label.

The present invention is also concerned with method of identifying or diagnosing a human subject with a pathology associated with extracellular trap formation or release from cells comprising administering a labelled antibody or antigen binding fragment thereof that specifically binds to a citrullinated epitope on deiminated human histone 2A and/or histone 4 to said subject, and diagnosing said pathology based on the localisation of the labelled antibody or antigen binding fragment in the human subject. The present invention is also concerned with method of identifying or diagnosing a human subject with a NET-associated pathology comprising administering a labelled antibody or antigen binding fragment thereof that specifically binds to a citrullinated epitope on deiminated human histone 2A and/or histone 4 to said subject, and diagnosing said pathology based on the localisation of the labelled antibody or antigen binding fragment in the human subject. The present invention is also concerned with method of identifying or diagnosing a human subject with an EET-associated pathology comprising administering a labelled antibody or antigen binding fragment thereof that specifically binds to a citrullinated epitope on deiminated human histone 2A and/or histone 4 to said subject, and diagnosing said pathology based on the localisation of the labelled antibody or antigen binding fragment in the human subject.

The present invention is also concerned with a method of identifying or diagnosing a human subject with a pathology associated with extracellular trap formation or release from cells comprising acquiring an image of the human subject to whom an amount of a labelled antibody or antigen binding fragment thereof that specifically binds to a citrullinated epitope on deiminated human histone 2A and/or histone 4 has been administered, wherein diagnosing said pathology is based on the localisation of the labelled antibody or antigen binding fragment in the image. The present invention is also concerned with a method of identifying or diagnosing a human subject with a NET-associated pathology comprising acquiring an image of the human subject to whom an amount of a labelled antibody or antigen binding fragment thereof that specifically binds to a citrullinated epitope on deiminated human histone 2A and/or histone 4 has been administered, wherein diagnosing said pathology is based on the localisation of the labelled antibody or antigen binding fragment in the image. The present invention is also concerned with a method of identifying or diagnosing a human subject with an EET-associated pathology comprising acquiring an image of the human subject to whom an amount of a labelled antibody or antigen binding fragment thereof that specifically binds to a citrullinated epitope on deiminated human histone 2A and/or histone 4 has been administered, wherein diagnosing said pathology is based on the localisation of the labelled antibody or antigen binding fragment in the image.

In a preferred embodiment of the invention, the labelled antibody is radioactively labelled. The amount of labelled antibody or antigen binding fragment thereof administered to the human subject is sufficient to allow imaging of the location of said labelled antibody or antigen binding fragment thereof in the human subject. The precise amount, or dosage, of labelled antibody or antigen binding fragment thereof administered to said human subject will depend on factors such as body weight of the human subject. Calculation of a suitable amount, or dosage, is standard practice for health practitioners carrying out the methods described herein.

In an embodiment of the invention, identification or diagnosis of the human subject with a pathology associated with extracellular trap formation or release from cells indicates that said human subject is likely to respond to a therapeutic agent that inhibits the formation of extracellular traps. In an embodiment of the invention, identification or diagnosis of the human subject with a NET-associated pathology or EET-associated pathology indicates that said human subject is likely to respond to a therapeutic agent that inhibits the formation of NETs, or EETs, respectively. In a preferred embodiment of the invention, said therapeutic agent is an antibody or binding fragment therefor described herein.

In an embodiment of the invention, the human subject identified or diagnosed with a pathology associated with extracellular trap formation or release from cells is subsequently treated with an agent that inhibits the formation of extracellular traps. In an embodiment of the invention, the human subject identified or diagnosed with a pathology associated with extracellular trap formation or release from cells is subsequently treated with an agent that inhibits the formation of extracellular traps, and said agent is an antibody or binding fragment thereof described herein. In an embodiment of the invention, the human subject identified or diagnosed with a NET-associated pathology is subsequently treated with an agent that inhibits the formation of NETs. In an embodiment of the invention, the human subject identified or diagnosed with an EET-associated pathology is subsequently treated with an agent that inhibits the formation of EETs. In an embodiment of the invention, the human subject identified or diagnosed with a NET-associated pathology or EET-associated pathology is subsequently treated with an agent that inhibits the formation of NETs or EETs, respectively, and said agent is an antibody or binding fragment thereof described herein.

In certain embodiments, the diagnostic methods described herein are used to select human subjects that are suitable for treatment with the antibodies or binding fragments thereof described herein. In certain embodiments, the invention encompasses a method of treating a human subject with a pathology associated with extracellular trap formation or release from cells, comprising administering an antibody or antigen binding fragment thereof that specifically binds to a citrullinated epitope on deiminated human histone 2A and/or histone 4 to said subject, wherein said subject has been diagnosed with a pathology associated with extracellular trap formation or release from cells in accordance with a diagnostic method described herein.

The present invention is also concerned with a method of determining the effectiveness of a treatment carried out on a human subject with a pathology associated with extracellular trap formation or release from cells, said method comprising acquiring an image of the human subject to whom an amount of a labelled antibody or antigen binding fragment thereof that specifically binds to a citrullinated epitope on deiminated human histone 2A and/or histone 4 has been administered, wherein determining the effectiveness of said treatment is based on the presence and/or localisation and/or quantification of the labelled antibody or antigen binding fragment in the image.

Effectiveness of treatment can be determined according to the knowledge of the skilled person. For example, effectiveness of treatment can determined based on the presence or absence of the labelled antibody or antigen binding fragment in an image taken from said human subject. Effectiveness of treatment can determined based on the localisation of the labelled antibody or antigen binding fragment in an image taken from said human subject. Effectiveness of treatment can determined based on the quantification of the labelled antibody or antigen binding fragment present in an image taken from said human subject. An effective treatment would be reflected in a reduction in the amount of labelled antibody or antigen binding fragment present in an image taken from said human subject, relative to a baseline image taken before treatment began.

In a preferred embodiment of the invention, the pathology associated with extracellular trap formation or release from cells is a NET-associated pathology or EET-associated pathology. In a preferred embodiment, said treatment comprises administration to the human subject an antibody or antigen binding fragment thereof that specifically binds to a citrullinated epitope on deiminated human histone 2A and/or histone 4 to said subject. In a preferred embodiment, said antibody or antigen binding fragment thereof is an antibody or antigen binding fragment thereof as described herein.

### Diseases to be Identified or Diagnosed

The present invention relates to antibodies or binding fragments thereof that specifically bind to a citrullinated epitope on deiminated human histone 2A and/or histone 4 for use in the identification or diagnosis of diseases associated with extracellular trap (ET) formation or release from cells, such as Macrophage Extracellular Trap (MET)-associated pathologies, Mast Cell Extracellular Trap (MCET)-associated pathologies, Monocyte derived Extracellular Trap-associated pathologies, Neutrophil Extracellular Trap (NET)-associated pathologies or Eosinophil Extracellular Trap (EET)-associated pathologies. Extracellular traps (ETs) are thread-like structures of decondensed DNA decorated with proteins from granules and cytoplasm and/or proteins from the cytoplasm.

The antibodies or binding fragments thereof of the present invention, or the pharmaceutical compositions as defined herein, are particularly suited for use in the identification or diagnosis of pathologies associated with citrullination, i.e. pathologies associated with a citrullinated epitope on deiminated human histone 2A and/or histone 4, such as pathologies associated with extracellular trap formation or release from cells, Macrophage Extracellular Trap (MET)-associated pathologies, Mast Cell Extracellular Trap (MCET)-associated pathologies, Monocyte derived Extracellular Trap-associated pathologies,NET-associated pathologies and inflammatory conditions and/or EET-associated pathologies. Said citrullinated epitope on deiminated human histone 2A and/or histone 4 is present in the decondensed DNA found in extracellular traps.

A pathology associated with citrullination can be defined as any disease or condition where citrullination is associated with the pathological state of the disease or condition. Whether or not citrullination plays a role in the pathogenesis of the disease, may be easily determined by a skilled person using routine tests available in the art. For example, these diseases may be characterized by the presence of an abnormal level of citrullinated proteins in affected or disease-related tissue. Such may be accomplished by an immunological test such as a Western blot or an ELISA wherein the affected tissue is used as an antigen and citrullination of that antigen may be detected with the aid of an anti-citrulline antibody as described herein. Alternatively, a person skilled in the art can use Proteomic applications such as mass spectrometry analysis to compare the level and type of citrullination in a diseased versus healthy tissue from affected patients.

Macrophage Extracellular Trap (MET)-associated pathologies, Mast Cell Extracellular Trap (MCET)-associated pathologies and Monocyte derived Extracellular Trap-associated pathologies can be considered as pathologies associated with citrullination. These pathologies can be defined as a disease or condition where the formation of extracellular traps (ET) and ETosis is associated with the pathological state of the disease or condition. Whether or not ET formation and ETosis plays a role in the pathogenesis of the disease may be easily determined by a skilled person using routine tests available in the art. For example, these diseases may be characterized by the presence of Macrophage Extracellular Traps, Mast Cell Extracellular Traps or Monocyte derived Extracellular Traps in the relevant tissues or blood compartment.

Examples of MET-associated pathologies can be found in Wu et al 2021 (Br J Pharmacol. 178:3783-3796) and Pertiwi et al 2019 (J Pathol. 247:505-512), both hereby incorporated by reference. MET-associated pathologies can include liver ischemia, liver reperfusion injury, coronary heart disease and atherothrombosis.

Examples of Mast Cell Extracellular Trap (MCET)-associated pathologies can be found in Pertiwi et al 2019 (J Pathol. 247:505-512), hereby incorporated by reference. Mast Cell Extracellular Trap (MCET)-associated pathologies can include coronary heart disease and atherothrombosis.

Examples of Monocyte derived Extracellular Trap-associated pathologies can be found in Schultz et al 2019 (Syst. Biol. Rep. Med. 65:357-366) and Granger et al. 2017 (JLB 102:775-781) both hereby incorporated by reference. Monocyte derived Extracellular Trap-associated pathologies can include pathologic contexts where monocytes are implicated, such as infertility, motility of human spermatozoa, inflammatory disorders, and thrombosis.

NET-associated pathologies can be considered as pathologies associated with citrullination. NET-associated pathologies can be defined as a disease or condition where the formation of NETs and NETosis is associated with the pathological state of the disease or condition. Whether or not NET formation and NETosis plays a role in the pathogenesis of the disease may be easily determined by a skilled person using routine tests available in the art. For example, these diseases may be characterized by the presence of NETs in the relevant tissues.

The invention therefore relates to antibodies or binding fragments thereof for use in the identification or diagnosis of NET-associated pathologies.

Examples of NET-associated pathologies include inflammatory conditions or diseases, ocular inflammatory diseases, cardiovascular diseases, respiratory diseases, wound healing, skin diseases, autoimmune diseases, cancer, and organ-health after transplant.

“Inflammatory Conditions” or Inflammatory diseases” refers to any of a number of conditions or diseases, which are characterized by vascular changes: edema and infiltration of neutrophils (e.g., acute inflammatory reactions); infiltration of tissues by mononuclear cells; tissue destruction by inflammatory cells, connective tissue cells and their cellular products; and attempts at repair by connective tissue replacement (e.g., chronic inflammatory reactions). Such diseases are for instance inflammatory arthritis, including rheumatoid arthritis and osteoarthritis, SLE, lupus, sepsis, vasculitis, multiple sclerosis, psoriatic arthritis, psoriasis, hidradenitis suppurativa, Alzheimer's disease, autoimmune hepatitis, juvenile idiopathic arthritis, myositis (polymyositis and dermatomyositis), Sjögren's disease, spondyloarthropathy, multiple system atrophy, heart failure, atherosclerosis, Parkinson's disease, Lewy body dementia, idiopathic pulmonary fibrosis, dry eye disease, uveitis, nongranulomatous uveitis, granulomatous uveitis, dermatitis, atopic dermatitis, and lung diseases such as asthma, acute respiratory distress syndrome (ARDS) including but not limited to coronavirus induced ARDS, COPD and bronchitis, thrombotic diseases and cardiovascular diseases. Nongranulomatous uveitis can be associated with neutrophil dominant inflammation, granulomatous uveitis can be associated with macrophage dominant inflammation.

NETs play a role in autoimmune diseases pathology, including RA, SLE and vasculitis.

In a preferred embodiment, the diseases to be identified or diagnosed are NET-associated pathologies such as systemic lupus erythematosus (SLE), idiopathic inflammatory myopathy, lupus, sepsis, thrombosis, vasculitis, small-vessel vasculitis (SVV), antiphospholipid syndrome (APS), inflammatory arthritis, rheumatoid arthritis and osteoarthritis, psoriasis, dermatomyositis, polymyositis, inclusion body myositis, hidradenitis suppurativa, Alzheimer's disease, autoimmune hepatitis, juvenile idiopathic arthritis, ulcerative colitis, myositis (polymyositis and dermatomyositis), Sjögren's disease, Anti-phospholipid Syndrome, Bechet's disease, spondylitis, spondyloarthropathy, multiple system atrophy, Parkinson's disease, Lewy body dementia, heart failure with preserved ejection fraction, heart failure with reduced ejection fraction, asthma, allergic rhinovirus exacerbated asthma, allergic asthma, cystic fibrosis, fibrosis and idiopathic pulmonary fibrosis, idiopathic pulmonary fibrosis, atherosclerosis, dry eye disease, uveitis, nongranulomatous uveitis, granulomatous uveitis, dermatitis, atopic dermatitis, inflammatory bowel disease, COPD, bronchitis, or other NET-associated pathologies such as wound healing in diabetes, cancer, cancer metastasis, and transplant organ health in vivo or ex vivo.

In a preferred embodiment, the diseases to be identified or diagnosed are inflammatory conditions such as systemic lupus erythematosus (SLE), idiopathic inflammatory myopathy, lupus, sepsis, thrombosis, vasculitis, small-vessel vasculitis (SVV), antiphospholipid syndrome (APS), inflammatory arthritis, rheumatoid arthritis and osteoarthritis, psoriasis, dermatomyositis, polymyositis, inclusion body myositis, hidradenitis suppurativa, Alzheimer's disease, autoimmune hepatitis, juvenile idiopathic arthritis, ulcerative colitis, myositis (polymyositis and dermatomyositis), Sjögren's disease, Anti-phospholipid Syndrome, Bechet's disease, spondylitis, spondyloarthropathy, multiple system atrophy, Parkinson's disease, Lewy body dementia asthma, heart failure with preserved ejection fraction, heart failure with reduced ejection fraction, asthma, allergic rhinovirus exacerbated asthma, allergic asthma, cystic fibrosis, fibrosis and idiopathic pulmonary fibrosis, idiopathic pulmonary fibrosis, atherosclerosis, dry eye disease, uveitis, nongranulomatous uveitis, granulomatous uveitis, dermatitis, atopic dermatitis, inflammatory bowel disease, COPD or bronchitis.

In one preferred embodiment, the NET-associated pathology is a tauopathy. In a particularly preferred embodiment, the disorder is Parkinson's Disease.

In one preferred embodiment, the NET-associated pathology is identified or diagnosed at an early stage. In a preferred embodiment, the NET-associated pathology is early-stage rheumatoid arthritis.

The methods disclosed herein may also be for the identification or diagnosis of any disease or condition which includes an EET-associated pathology. An EET-associated pathology typically means a pathology which is mediated in whole or in part by the formation of EETs. Such a pathology is typically present in any disease or condition which is mediated in whole or in part, or preferably which is mediated primarily, by eosinophils. Such diseases or conditions may be described herein as eosinophilic or eosinophil-associated. Therefore, put another way, the methods disclosed herein may be for the identification or diagnosis of an eosinophilic disease or condition.

An eosinophilic disease or condition may be defined as a disease or condition in which eosinophils are present in elevated numbers in the tissue or organ affected, relative to the same tissue or organ in a healthy individual. Eosinophilic diseases and conditions may include: an eosinophilic disease or condition of the skin; a respiratory eosinophilic disease or condition; a gastro-intestinal eosinophilic disease or condition; an allergic disease or condition; or a helminth, fungal, viral, or bacterial infection.

Eosinophilic diseases or conditions of the skin include Bullous Pemphigoid (PB), Atopic dermatitis (AD) and Chronic spontaneous Urticaria (CSU), allergic contact dermatitis, and eosinophilic cellulitis (also called Well's syndrome).

Respiratory eosinophilic diseases or conditions include Eosinophilic Asthma, Nasal Polyps, Chronic RhinoSinusitis with Nasal Polyposis (CRSwNP), Allergic sinusitis, Allergic rhinitis, Allergic bronchopulmonary aspergillosis (a fungal infection), Eosinophilic chronic rhinosinusitis, Tropical pulmonary eosinophilia (typically a respiratory Helminth infection).

Gastro-intestinal eosinophilic diseases or conditions include Eosinophilic Esophagitis (EoE), Eosinophilic gastritis (stomach—EG), Eosinophilic gastroenteritis (stomach and small intestine—EGE), Eosinophilic enteritis (small intestine), Eosinophilic colitis (large intestine—EC), and a gastro-intestinal helminth infection such as Ascariasis or Trichinosis.

Other eosinophilic diseases or conditions include HyperEosinophilic Syndrome (HES—affects blood and various organs), Eosinophilic Granulomatosis with PolyAngitis (EGPA—affects various organs including blood vessels) and Eosinophilic otitis media (EOM—affects the middle ear), and Drug Reaction with Eosinophilic & Systemic Symptoms (DRESS—affects various organs).

In one preferred embodiment, the disease to be identified or diagnosed is arteriosclerosis. In another embodiment vasculitis is identified or diagnosed.

The methods disclosed herein may be for the identification or diagnosis of any of the above-listed eosinophilic diseases or conditions. Particularly preferred eosinophilic diseases or conditions include those in which the presence of EETs has been directly confirmed. Such disease and conditions include but are not limited to: Bullous Pemphigoid, Atopic dermatitis, allergic contact dermatitis, Eosinophilic Asthma, Chronic RhinoSinusitis with Nasal Polyposis (CRSwNP), Allergic sinusitis, Allergic bronchopulmonary aspergillosis, Eosinophilic chronic rhinosinusitis, Eosinophilic Esophagitis (EoE), HyperEosinophilic Syndrome (HES), Eosinophilic Granulomatosis with PolyAngitis (EGPA), Eosinophilic otitis media (EOM), and Drug Reaction with Eosinophilic & Systemic Symptoms (DRESS).

The most preferred eosinophilic diseases or conditions are those in which a correlation between EETs and disease incidence and/or severity has been directly observed. Such disease and conditions include but are not limited to: Eosinophilic Asthma, Chronic RhinoSinusitis with Nasal Polyposis (CRSwNP), Eosinophilic chronic rhinosinusitis, and Eosinophilic otitis media (EOM).

The presence of EETs and/or a role for eosinophils in diseases such as those discussed above is well-established in the art. See for example: Williams, T. L et al. (2020). “NETs and EETs, a Whole Web of Mess”. Microorganisms, 8(12), 1925 and Mukherjee, M., et al. (2018). Eosinophil Extracellular Traps and Inflammatory Pathologies-Untangling the Web!. Frontiers in immunology, 9, 2763. The invention is suitable for the identification or diagnosis of any disease recited in these documents, which are incorporated by reference.

The methods disclosed herein may also be for the identification or diagnosis of an EET-associated pathology in a disease or condition which is only partly mediated by eosinophils. For example, diseases such as Chronic Obstructive Pulmonary Disease (COPD), Crohn's disease, ulcerative colitis, dermatitis herpetiformis, thrombosis, and atherosclerosis may exhibit multiple pathologies caused by multiple cell types, and so may not be defined as “eosinophilic”. However, they may nonetheless exhibit EET-associated pathology and thus be identified or diagnosed by the methods disclosed herein.

In one preferred embodiment, the present invention is employed to identify or diagnose a lung disorder. In one embodiment, the antibody or binding fragment is any of those described herein. In a preferred embodiment, the lung disorder may be an inflammatory lung disorder. In one embodiment, the lung disorder is one characterized by an influx of inflammatory cells to the lung compared to a healthy subject without the disorder. For example, the condition may be in one embodiment characterized by an influx of white blood cells to the lung. In one embodiment, the lung disorder is characterized by an influx of granulocytes to the lung, in particular eosinophils and/or neutrophils to the lung.

In one embodiment, the lung condition is characterized by the subject showing poor symptom responsiveness to corticosteroids. In particular, in one embodiment, the approach provided is used to identify or diagnose a subject with a lung condition showing poor responsiveness to dexamethasone.

The methods may be used to identify or diagnose any suitable lung disorder, particularly an inflammatory lung disorder. In one embodiment, the lung disorder is selected from COPD, bronchitis, emphysema, cystic fibrosis, fibrosis and idiopathic pulmonary fibrosis, and asthma. In a preferred embodiment, the condition is asthma. It may be that the subject has severe asthma. In one particularly preferred embodiment, the lung disorder may be allergic asthma. In one preferred embodiment, the lung disorder is allergic asthma involving house dust mite allergy. In one embodiment, the lung disorder is asthma characterised by the presence of a raised number of eosinophils and/or neutrophils. In one embodiment a method of the present invention may be used to identify or diagnose a lung condition with increased numbers of infiltrating eosinophils. In another embodiment, a method of the present invention may be used to identify or diagnose a lung condition with increased numbers of infiltrating neutrophils. In another embodiment, the subject has increased numbers of infiltrating eosinophils and neutrophils. In one embodiment the subject may have neutrophilic asthma. In another embodiment, the subject may have eosinophilic asthma. In one embodiment, the subject may have type 2 asthma. In another embodiment, the subject may have non-type 2 asthma.

In one preferred embodiment, the EET-associated pathology is identified or diagnosed at an early stage.

Typically, antibodies or compositions comprising them are administered to a subject already suffering from a disorder or condition, in an amount sufficient to identify or diagnose the condition. Effective amounts for a given purpose will depend on the severity of the disease or injury as well as the weight and general state of the subject. As used herein, the term “subject” includes any human.

### Antibodies

The present invention relates to antibodies or binding fragments thereof that specifically bind to a citrullinated epitope on deiminated human histone 2A and/or histone 4 and in particular to such antibodies or binding fragments thereof to be employed in identifying or diagnosing a pathology associated with extracellular trap formation or release from cells, such as a Macrophage Extracellular Trap (MET)-associated pathology, Mast Cell Extracellular Trap (MCET)-associated pathology, Monocyte derived Extracellular Trap-associated pathology, Neutrophil Extracellular Trap associated pathology (a NET-associated pathology) or Eosinophil Extracellular Trap associated pathology (a EET-associated pathology). The present section provides examples of possible antibodies or binding fragments thereof that are provided for use as set out herein and also to be employed in the methods set out herein. For the sake of brevity though, the present section will simply refer to antibodies, but both the antibodies for use as set out herein and methods comprising their administration are provided.

Citrulline is an amino acid that is not incorporated into proteins during normal translation, however, it may be generated by post-translational modification of an arginine residue by enzymes such as Peptidylarginine deiminases (PAD); (EC 3.5.3.15). In mammals (humans, mice and rats), five PAD isotypes (PAD1-PAD6; ‘PAD4’ and ‘PAD5’ are used for the same isotype), each encoded by a distinct gene, have been identified thus far.

Citrullination of histone 2A and/or histone 4 is associated with the formation of NETs. The downstream pathological effects of NET formation can be numerous. For example, there can be autoantigen exposure to the extracellular space and the subsequent production of pathological autoantibodies by the subject. NET-derived histones can be toxic to the vascular wall and organs leading to vascular damage and organ failure. NETs can lead to the formation of autoantigen/autoantibody immune complexes, which enhance further inflammation, in for example the kidney of SLE patients. NETs are also involved in metastasis in cancer progression.

Any suitable antibody or binding fragment thereof that specifically binds to a citrullinated epitope on deiminated human histone 2A and/or histone 4 may be employed in the present invention. Deimination of human histone 2A and 4 can be carried out by enzymes such as PADs, for example PAD2 and PAD4. In a specific embodiment, the antibodies or binding fragments thereof according to the invention specifically bind to a citrullinated epitope on deiminated human histone 2A and/or histone 4, wherein the epitope comprises a peptide selected from the group consisting of SEQ ID NOs: 18, 19, 20, 21 and 22. The antibodies or binding fragments thereof may also bind to epitopes comprising the peptides of SEQ ID NO: 53 or 54.

The term “antibodies”, “antibody” or “binding fragment thereof” as used herein refers to a structure, preferably a protein or polypeptide structure, capable of specific binding to a target molecule often referred to as “antigen”. The antibody molecule as employed herein refers to an antibody or binding fragment thereof. In a particularly preferred embodiment, the term ‘antibody’ as used herein generally relates to intact (whole) antibodies i.e. comprising the elements of two heavy chains and two light chains. The antibody may comprise further additional binding domains for example as per the molecule DVD-Ig as disclosed in WO 2007/024715, or the so-called (FabFv)₂Fc described in WO2011/030107. Thus ‘antibody’ as employed herein includes mono-, bi-, tri- or tetra-valent full-length antibodies. Reference to an “antibody” herein specifically encompasses an antigen binding fragment being employed, unless it is clear from the context used that is not the case.

An antibody or binding fragment thereof may be selected from the group consisting of single chain antibodies, single chain variable fragments (scFvs), variable fragments (Fvs), fragment antigen-binding regions (Fabs), recombinant antibodies, monoclonal antibodies, fusion proteins comprising the antigen-binding domain of a native antibody or an aptamer, single-domain antibodies (sdAbs), also known as VHH antibodies, nanobodies (Camelid-derived single-domain antibodies), shark IgNAR-derived single-domain antibody fragments called VNAR, diabodies, triabodies, Anticalins, aptamers (DNA or RNA) and active components or fragments thereof.

Multi-valent antibodies may comprise multiple specificities e.g. bispecific or may be monospecific.

In one embodiment, rather than a full-length antibody an antigen-binding fragment thereof may be employed. Binding fragments of antibodies include single chain antibodies (i.e. a full-length heavy chain and light chain); Fab, modified Fab, Fab′, modified Fab′, F(ab′)2, Fv, Fab-Fv, Fab-dsFv, single domain antibodies (e.g. VH or VL or VHH), scFv, mono-, bi-, tri- or tetra-valent antibodies, Bis-scFv, diabodies, tribodies, triabodies, tetrabodies and epitope-binding fragments of any of the above (see for example Holliger P and Hudson P J, 2005, Nat. Biotechnol., 23: 1126-1136; Adair J R and Lawson A D G, 2005, Drug Design Reviews—Online, 2, 209-217). The methods for creating and manufacturing these antibody fragments are well known in the art (see for example Verma R et al., 1998, J. Immunol. Methods, 216, 165-181). The Fab-Fv format was first disclosed in WO2009/040562 and the disulphide-stabilised versions thereof, the Fab-dsFv was first disclosed in WO2010/035012. Other antibody fragments for use in the present invention include Fab and Fab′ fragments.

In one embodiment, the antibody is selected from a full-size antibody, Fab, F(ab′)2, single-chain Fv fragment, single-domain VHH, Single domain VH or single domain VL.

IgG1 (e.g. IgG1/kappa) antibodies having an IgG1 heavy chain and a light chain may advantageously be used in the invention. However, other human antibody isotypes are also encompassed by the invention, including IgG2, IgG3, IgG4, IgM, IgAQ1, IgA2, IgAsec, IgD and IgE in combination with a kappa or lambda light chain. Also, all animal-derived antibodies of various isotypes can be used in the invention.

The term: “specifically binds to citrulline” or “specifically binds to a citrullinated epitope” in this context means that the antibody or binding fragment thereof binds to a structure such as a peptide containing a citrulline residue whereas the antibody or binding fragment thereof binds less strongly or preferably not at all with the same structure containing an arginine residue instead of the citrulline residue. The term peptide should be interpreted as a structure that is capable of presenting the citrulline residue in the correct context for immunoreactivity with the antibodies or binding fragments thereof as described herein, preferably in the same context as it appears in the human or animal body, preferably in the context of a native polypeptide.

The antibodies or binding fragments thereof suitable for use in the methods of the invention specifically bind to a citrullinated epitope on deiminated human histone 2A and/or histone 4. The binding of antibodies or binding fragments thereof to a citrullinated epitope on deiminated human histone 2A and/or histone 4 blocks ET formation. Citrullination of histones is associated with the formation of ETs. Blocking of ET formation can be total or partial. For example, the antibody or binding fragment thereof of the invention may reduce ET formation from 10 to 50%, at least 50% or at least 70%, 80%, 90%, 95% or 99%. ET blocking can be measured by any suitable means, for example by measuring ETosis in vitro (Pertiwi et al 2019, J Pathol. 247:505-512 which describes use of an anti-CitH3 antibody in combination with a cell specific antibody to determine the presence of ETs).

The antibodies or binding fragments thereof of the invention specifically bind to a citrullinated epitope on deiminated human histone 2A and/or histone 4. The binding of antibodies or binding fragments thereof to a citrullinated epitope on deiminated human histone 2A and/or histone 4 blocks NET formation. Citrullination of histones is associated with the formation of NETs. Blocking of NET formation can be total or partial. For example, the antibody or binding fragment thereof of the invention may reduce NET formation from 10 to 50%, at least 50% or at least 70%, 80%, 90%, 95% or 99%. NET blocking can be measured by any suitable means, for example by measuring NETosis in vitro (Kraaij T et al., 2016, Autoimmun. Rev. 15, 577-584).

The antibodies or binding fragments thereof suitable for us in the methods of the invention specifically bind to a citrullinated epitope on deiminated human histone 2A and/or histone 4. The binding of antibodies or binding fragments thereof to a citrullinated epitope on deiminated human histone 2A and/or histone 4 blocks EET formation. Citrullination of histones is associated with the formation of EETs.

Blocking of EET formation can be total or partial. For example, the antibody or binding fragment thereof may reduce EET formation from 10 to 50%, at least 50% or at least 70%, 80%, 90%, 95% or 99%. EET blocking can be measured by any suitable means, for example by measuring EETosis in vitro (Fukuchi et al., “How to detect eosinophil ETosis (EETosis) and extracellular traps”; Allergology International, Volume 70, Issue 1, 2021, Pages 19-29).

The terms “binding activity” and “binding affinity” are intended to refer to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule. Typically, the Kd for the antibody with respect to the target will be 2-fold, preferably 5-fold, more preferably 10-fold less than the Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci M S and Cacheris W P (1984, Byte, 9, 340-362). For example, the Kd may be established using a double-filter nitrocellulose filter binding assay such as that disclosed by Wong I and Lohman™ (1993, Proc. Natl. Acad. Sci. USA, 90, 5428-5432) or for example, by using Octet surface plasmon resonance.

One method for the evaluation of binding affinity for deiminated human histone 2A and/or histone 4 is by ELISA. Other standard assays to evaluate the binding ability of ligands such as antibodies towards targets are known in the art, including for example, Western blots, RIAs, and flow cytometry analysis. The binding kinetics (e.g. binding affinity) of the antibody also can be assessed by standard assays known in the art, such as surface plasmon resonance, for example by Biacore™ system analysis.

Preferably the antibody has a binding affinity for deiminated human histone 2A and/or histone 4 of 1 nM or less. Preferably the antibody of the invention has a binding affinity for deiminated human histone 2A and/or histone 4, and/or deiminated human histone H3 of 0.5 nM or less, 0.1 nM or less, 50 pM or less, 10 pM or less, 5 pM or less, 2 pM or less or 1 pM or less.

The antibody or binding fragment thereof may also be a fusion protein comprising the antigen-binding domain of a native antibody or an aptamer, such as an aptamer in the form of DNA or RNA.

Preferably the antibody or binding fragment thereof of the invention is a monoclonal antibody. Monoclonal antibodies are immunoglobulin molecules that are identical to each other and have a single binding specificity and affinity for a particular epitope. Monoclonal antibodies (mAbs) of the present invention can be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press).

The antibody or binding fragment thereof of the invention comprises a binding domain. A binding domain will generally comprise 6 CDRs (3 in case of VHH), three from a heavy chain and three from a light chain. In one embodiment the CDRs are in a framework and together form a variable region or domain. Thus, in one embodiment an antibody or binding fragment comprises a binding domain specific for the antigen comprising a light chain variable region or domain and a heavy chain variable region or domain.

The residues in antibody variable domains are conventionally numbered according to IMGT (http://www.imgt.org). This system is set forth in Lefranc M P (1997, J, Immunol. Today, 18, 509). This numbering system is used in the present specification except where otherwise indicated.

The IMGT residue designations do not always correspond directly with the linear numbering of the amino acid residues. The actual linear amino acid sequence may contain fewer or additional amino acids than in the strict IMGT numbering corresponding to a shortening of, or insertion into, a structural component, whether framework or CDR, of the basic variable domain structure. The correct IMGT numbering of residues may be determined for a given antibody by alignment of residues of homology in the sequence of the antibody with a “standard” IMGT numbered sequence.

The CDRs of the heavy chain variable domain are located at residues 27-38 (CDR1 of VH), residues 56-65 (CDR2 of VH) and residues 105-117 (CDR3 of VH) according to the IMGT numbering system.

The CDRs of the light chain variable domain are located at residues 27-38 (CDR1 of VL), residues 56-65 (CDR2 of VL) and residues 105-117 (CDR3 of VL) according to the IMGT numbering system.

The antibodies or binding fragments thereof of the present invention are set out herein by the primary amino acid sequence of their CDR regions. The antibodies or binding fragments thereof of the present invention are disclosed herein by the primary amino acid sequence of their heavy and light chains.

In a particularly preferred embodiment, a modified CDR1 of the VL of an antibody or binding fragment thereof that specifically is employed to provide improved properties to the antibody or binding fragment thereof over an antibody or binding fragment thereof comprising an unmodified version of CDR1 of the VL. In one embodiment, the unmodified CDR1 of the VL of the antibody used to derive such a modified antibody comprises or consists of the amino acid sequences QSLLDSDGKTY (SEQ ID NO: 36) or QSLVDSDGKTY (SEQ ID NO: 37).

The modified CDR1 of the VL chain of the antibody or binding fragment thereof preferably comprises or consists of the amino acid sequence QSL-X₁-D-X₂-D-X₃-KTY, wherein X₁ is V or L, X₂ is T, S, A or N and X₃ is G or A, provided that the amino acid sequence is not QSLLDSDGKTY (SEQ ID NO: 36) or QSLVDSDGKTY (SEQ ID NO: 37). The modified CDR1 of the VL chain of the antibody or binding fragment thereof of shows reduced isomerisation, in comparison with the unmodified CDR1 of SEQ ID NO: 36 or 37, but maintains the binding properties of the unmodified CDR1.

The amino acid sequences of the CDRs for the VH of a particular antibody or binding fragment thereof of the invention are shown in SEQ ID NOs: 1, 2 and 3. The CDRs 2 and 3 for the VL are shown in SEQ ID NOs: 4 and 5.

The amino acid sequences of the VH and VL of a particular antibody or binding fragment thereof of a particularly preferred antibody are given in SEQ ID NOs: 11 and 13. The CDRs for the VH are shown in SEQ ID NOs: 1, 2 and 3. The CDRs for the VL are shown in SEQ ID NOs: 6, 4 and 5.

The amino acid sequences of the VH and VL of another preferred antibody or binding fragment thereof are given in SEQ ID NOs: 11 and 14. The CDRs for the VH are shown in SEQ ID NOs: 1, 2 and 3. The CDRs for the VL are shown in SEQ ID NOs: 7, 4 and 5.

The amino acid sequences of the VH and VL of another preferred antibody or binding fragment thereof are given in SEQ ID NOs: 11 and 15. The CDRs for the VH are shown in SEQ ID NOs: 1, 2 and 3. The CDRs for the VL are shown in SEQ ID NOs: 8, 4 and 5.

The amino acid sequences of the VH and VL of another preferred antibody or binding fragment thereof are given in SEQ ID NOs: 11 and 16. The CDRs for the VH are shown in SEQ ID NOs: 1, 2 and 3. The CDRs for the VL are shown in SEQ ID NOs: 9, 4 and 5.

The amino acid sequences of the VH and VL of another preferred antibody or binding fragment thereof are given in SEQ ID NOs: 11 and 17. The CDRs for the VH are shown in SEQ ID NOs: 1, 2 and 3. The CDRs for the VL are shown in SEQ ID NOs: 10, 4 and 5.

The amino acid sequences of the VH and VL of another preferred antibody or binding fragment are given in SEQ ID NOs: 12 and 13. The CDRs for the VH are shown in SEQ ID NOs: 1, 2 and 3. The CDRs for the VL are shown in SEQ ID NOs: 6, 4 and 5.

The amino acid sequences of the VH and VL of another preferred antibody or binding fragment thereof are given in SEQ ID NOs: 12 and 14. The CDRs for the VH are shown in SEQ ID NOs: 1, 2 and 3. The CDRs for the VL are shown in SEQ ID NOs: 7, 4 and 5.

The amino acid sequences of the VH and VL of another preferred antibody or binding fragment thereof are given in SEQ ID NOs: 12 and 15. The CDRs for the VH are shown in SEQ ID NOs: 1, 2 and 3. The CDRs for the VL chain are shown in SEQ ID NOs: 8, 4 and 5.

The amino acid sequences of the VH and VL of another preferred antibody or binding fragment are given in SEQ ID NOs: 12 and 16. The CDRs for the VH are shown in SEQ ID NOs: 1, 2 and 3. The CDRs for the VL are shown in SEQ ID NOs: 9, 4 and 5.

The amino acid sequences of the VH and VL of another preferred antibody or binding fragment thereof are given in SEQ ID NOs: 12 and 17. The CDRs for the VH are shown in SEQ ID NOs: 1, 2 and 3. The CDRs for the VL are shown in SEQ ID NOs: 10, 4 and 5.

In an embodiment, the antibody of the invention comprises the heavy chain variable domain amino acid sequence of SEQ ID NO: 11, the light chain variable domain amino acid sequence of SEQ ID NO: 16, a heavy chain constant region amino acid sequence comprising SEQ ID NO: 23 or 56, and the light chain constant region amino acid sequence of SEQ ID NO: 24.

In an embodiment, the antibody of the invention comprises the heavy chain variable domain amino acid sequence of SEQ ID NO: 11, the light chain variable domain amino acid sequence of SEQ ID NO: 16, the heavy chain constant region amino acid sequence of SEQ ID NO: 23 or 56, and the light chain constant region amino acid sequence of SEQ ID NO: 24.

An antibody or binding fragment thereof employed in the invention may comprise one or more of the CDR sequences of any one of the specific antibodies as described above, except that the CDR1 of the VL is always present as either comprising or consisting of the amino acid sequence QSL-X₁-D-X₂-D-X₃-KTY, wherein X₁ is V or L, X₂ is T, S, A or N and X₃ is G or A, provided that the amino acid sequence is not QSLLDSDGKTY (SEQ ID NO: 36) or QSLVDSDGKTY (SEQ ID NO: 37), or either comprises or consists of SEQ ID NOs: 6, 7, 8, 9 or 10.

An antibody or binding fragment thereof employed in the invention may comprise one or more VH CDR sequences and alternatively or additionally one or more VL CDR sequences of said specific antibody, in addition to VL CDR1. An antibody or binding fragment thereof may comprise one, two or all three of the VH CDR sequences of a specific antibody or binding fragment thereof as described above and alternatively or additionally one, two or all three of the VL chain CDR sequences of said specific antibody or binding fragment thereof, including VL CDR1. An antibody or binding fragment thereof may comprises all six CDR sequences of a specific antibody or binding fragment as described above. By way of example, an antibody to be employed in the invention may comprise one of SEQ ID NO: 6, 7, 8, 9 or 10 and one or more of SEQ ID NOs: 1, 2, 3, 4 and 5.

In an embodiment of the invention, the modified CDR1 of the VL chain of the antibody or binding fragment thereof of the antibody employed comprises or consists of the amino acid sequence QSL-Z₁-Z₂-Z₃-Z₄-Z₅-KTY, wherein Z₁ is V or L, Z₂ is D or E, Z₃ is T, S, A or N, Z₄ is D, E, S or A and Z₅ is G or A, provided that the amino acid sequence is not QSLLDSDGKTY (SEQ ID NO: 36) or QSLVDSDGKTY (SEQ ID NO: 37). The modified CDR1 of the VL chain of the antibody or binding fragment thereof of the invention shows reduced isomerisation, in comparison with the unmodified CDR1 of SEQ ID NO: 36 or 37, but maintains the binding properties of the unmodified CDR1. The modified CDR1 of the VL chain of the antibody or binding fragment thereof may comprise or consist of in some embodiments SEQ ID NO: 6, 7, 8, 9, 10, 41, 42, 43, 44, 45, 46, 47, 48, 49, 50, 51 or 52. In an embodiment, the antibody may comprise one of SEQ ID NO: 6, 7, 8, 9, 10, 41, 42, 43, 44, 45, 46, 47, 48, 49, 50, 51 or 52, and one or more of SEQ ID NOs: 1, 2, 3, 4 and 5. In one embodiment, the antibody comprises one of SEQ ID NO: 6, 7, 8, 9, 10, 41, 42, 43, 44, 45, 46, 47, 48, 49, 50, 51 or 52, and all of SEQ ID NOs: 1, 2, 3, 4 and 5.

An antibody or binding fragment thereof to be employed in the invention may alternatively comprise a variant of one of these heavy chain variable domains or CDR sequences in CDR2 or 3 of the VL. For example, a variant may be a substitution, deletion or addition variant of any of the above amino acid sequences.

A variant antibody may comprise 1, 2, 3, 4, 5, up to 10, up to 20, up to 30 or more amino acid substitutions and/or deletions from the specific sequences and fragments discussed above, whilst maintaining the activity of the antibodies described herein. “Deletion” variants may comprise the deletion of, for example, 1, 2, 3, 4 or 5 individual amino acids or of one or more small groups of amino acids such as 2, 3, 4 or 5 amino acids. “Small groups of amino acids” can be defined as being sequential, or in close proximity but not sequential, to each other. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid, another aliphatic amino acid, another tiny amino acid, another small amino acid or another large amino acid. Some properties of the 20 main amino acids, which can be used to select suitable substituents, are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

Preferred “derivatives” or “variants” include those in which instead of the naturally occurring amino acid the amino acid, which appears in the sequence, is a structural analog thereof. Amino acids used in the sequences may also be derivatized or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variant antibodies have an amino acid sequence which has more than 60%, or more than 70%, e.g. 75 or 80%, preferably more than 85%, e.g. more than 90%, 95%, 96%, 97%, 98% or 99% amino acid identity to the VL and/or VH, or a fragment thereof, of an antibody disclosed herein. This level of amino acid identity may be seen across the full-length of the relevant SEQ ID NO sequence or over a part of the sequence, such as across 20, 30, 50, 75, 100, 150, 200 or more amino acids, depending on the size of the full-length polypeptide.

Preferably the variant antibodies comprise one or more of the CDR sequences as described herein.

In connection with amino acid sequences, “sequence identity” refers to sequences, which have the stated value when assessed using ClustalW (Thompson J D et al., 1994, Nucleic Acid Res., 22, 4673-4680) with the following parameters:

Pairwise alignment parameters—Method: slow/accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G, P, S, N, D, Q, E, K, R. Sequence identity at a particular residue is intended to include identical residues, which have simply been derivatized.

Thus, antibodies having specific VH and VL amino acid sequences and variants and fragments thereof, which maintain the function or activity of these VHs and VLs, may be employed in the present invention.

Accordingly, antibodies or binding fragments thereof may be employed comprising variants of the VH that retain the ability of specifically binding a citrullinated epitope on human deiminated human histone 2A and/or histone 4. A variant of the heavy chain may have at least 70%, at least 80%, at least 85%, at least 90%, at least 95%, at least 96%, at least 97%, at least 98% or at least 99% amino acid sequence identity to the unmodified VH. The variant of the VH may comprise a fragment of at least 7 amino acids of hVH22.101f or hVH22.101HC9 (SEQ ID NO: 11 and 12, respectively), wherein the antibody or binding fragment thereof retains the ability of being specifically reactive with a citrullinated epitope on deiminated human histone 2A and/or histone 4; or

a variant of hVH22.101f or hVH22.101HC9 (SEQ ID NO: 11 and 12, respectively) having at least 70% amino acid sequence identity to a sequence of hVH22.101f or hVH22.101HC9 (SEQ ID NO: 11 and 12, respectively), wherein the antibody or binding fragment thereof retains the ability of being specifically reactive with a citrullinated epitope on deiminated human histone 2A and/or histone 4.

A particularly preferred antibody of the invention is the CIT-013 antibody as described herein. The CIT-013 antibody comprises the CDRs of SEQ ID NOs 1, 2, 3, 4, 5 and 9. The CIT-013 antibody comprises the heavy chain variable domain amino acid sequence of SEQ ID NO: 11 and the light chain variable domain amino acid sequence of SEQ ID NO: 16. The CIT-013 antibody comprises the heavy chain constant region of SEQ ID NO: 23 or 56, and a light chain constant region of SEQ ID NO: 24.

### Labelling

In a preferred embodiment of the invention, the antibody or binding fragment thereof for use in the diagnostic methods described herein may be labelled. In a preferred embodiment, the antibody or binding fragment thereof of the invention may be linked (directly or indirectly) to another moiety. The other moiety may be a label. The other moiety may be a detectable label. The other moiety may be a chelator. The other moiety may be a chelator in combination with a label. The other moiety may be a binding moiety, such as an antibody or a polypeptide binding domain specific for a therapeutic target. In some embodiments the labelling of the antibody or binding fragment thereof is direct by conjugation of the antibody or binding fragment thereof to the label. In some embodiments the labelling of the antibody or binding fragment thereof is indirect, by first conjugating a chelator to the antibody or binding fragment thereof, with the chelator being subsequently labelled with a moiety such as a radionuclide. In preferred embodiments the detectable label or label is a fluorescent, an enzymatic, bioluminescent or a radioactive label. In a particularly preferred embodiment the detectable label or label is a radioactive label.

The moiety, label, detectable label or chelator may be directly attached, for example by chemical conjugation, to an antibody or binding fragment thereof for use in the invention. Methods of conjugating agents or labels to an antibody are known in the art. For example, random conjugation of bifunctional chelators for labelling with radiometals is usually performed to lysines or cysteines. Site-specific conjugation might be performed by directed cysteine modification using maleimides or via click-chemistry, for example using the catalyst-free strain-promoted azide-alkyne cycloaddition (SPAAC) reaction or inverse electron demand Diels-Alder (IEDDA) reaction. Other mechanisms for site-specific conjugation include amongst others enzyme mediated conjugation, glycan engineering or biotin-(strept)avidin interactions.

Antibodies or binding fragments thereof might be radioiodinated by direct electrophilic addition reactions to tyrosine and histidine residues, or indirect using radioiodination of a prosthetic group. Fluorination can be performed by direct or indirect nucleophilic and electrophilic substitution reactions.

In addition, carbodiimide conjugation (Bauminger S and Wilchek M, 1980, Methods Enzymol., 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to antibodies can also be used. For example, sodium periodate oxidation followed by reductive alkylation of appropriate reactants can be used, as can glutaraldehyde cross-linking or carbodiimide conjugation. However, it is recognised that, regardless of which method of producing a conjugate of the invention is selected, a determination must be made that the antibody maintains its targeting ability and that the functional moiety maintains its relevant function.

The antibody may be linked to a detectable label. By “detectable label” it is meant that the antibody is linked to a moiety which, when located at the target site following administration of the antibody into a patient, may be detected, typically non-invasively from outside the body and the site of the target located. Thus, the antibody may be useful in imaging and diagnosis and determining treatment response.

Typically, the label is or comprises a radioactive atom which is useful in imaging. Suitable radioactive atoms include but are not limited to 67Cu, 99mTc, 111In, 123I, 131I, 155Tb, 186Rh and 188Rh for imaging of gamma rays with SPECT or scintigraphic methods, or 11C, 18F, 43/44Sc, 52Mn, 64/66Cu, 86Y, 89Zr, 124I, 152Tb for imaging of positron emission by PET imaging. Other labels include, for example, spin labels for magnetic resonance imaging (MRI) such as 123I again, 131I, 111 In, 19F, 13C, 15N, 170, gadolinium, manganese or iron, or fluorescent labels such as 5-ALA, methylene blue, fluorescein, indocyanine green, cyanine red dyes, IRDye680RD, IRDye700DX, IRDye800CW. Clearly, the sufficient amount of the appropriate atomic isotopes must be linked to the antibody in order for the molecule to be readily detectable.

It is envisaged that the antibodies or binding fragments described herein are used in nuclear imaging techniques including SPECT Single photon emission computed tomography) or PET (positron emission tomography) imaging. It is envisaged that the antibodies or binding fragments described herein are used in magnetic resonance imaging or fluorescent imaging.

It is envisaged that the images taken of human subjects to whom a labelled antibody or antigen binding fragment thereof that specifically binds to a citrullinated epitope on deiminated human histone 2A and/or histone 4 have been administered are images taken from radionuclide scanning. It is envisaged that the images taken of human subjects to whom a labelled antibody or antigen binding fragment thereof that specifically binds to a citrullinated epitope on deiminated human histone 2A and/or histone 4 have been administered are images from SPECT scans or PET scans. It is envisaged that the images taken of human subjects to whom a labelled antibody or antigen binding fragment thereof that specifically binds to a citrullinated epitope on deiminated human histone 2A and/or histone 4 have been administered are images taken from SPECT/CT (computed tomography) scans or PET scans taken in coordination with CT or MRI (magnetic resonance imaging) scans.

### Pharmaceutical Compositions

The antibodies or binding fragments thereof may be in the form of a pharmaceutical composition comprising the antibodies or binding fragments thereof and a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intraocular, intramuscular, subcutaneous, intradermal or intraperitoneal administration (e.g. by injection or infusion). In certain embodiments, a pharmaceutically acceptable carrier comprises at least one carrier selected from the group consisting of a co-solvent solution, liposomes, micelles, liquid crystals, nanocrystals, nanoparticles, emulsions, microparticles, microspheres, nanospheres, nanocapsules, polymers or polymeric carriers, surfactants, suspending agents, complexing agents such as cyclodextrins or adsorbing molecules such as albumin, surface active particles, and chelating agents. In further embodiments, a polysaccharide comprises hyaluronic acid and derivatives thereof, dextran and derivatives thereof, cellulose and derivatives thereof (e.g. methylcellulose, hydroxy-propylcellulose, hydroxy-propylmethylcellulose, carboxymethylcellulose, cellulose acetate phthalate, cellulose acetate succinate, cellulose acetate butyrate, hydroxypropylmethyl-cellulose phthalate), chitosan and derivative thereof, [beta]-glucan, arabinoxylans, carrageenans, pectin, glycogen, fucoidan, chondrotin, dermatan, heparan, heparin, pentosan, keratan, alginate, cyclodextrins, and salts and derivatives, including esters and sulfates, thereof.

Preferred pharmaceutically acceptable carriers comprise aqueous carriers or diluents. Examples of suitable aqueous carriers that may be employed in the pharmaceutical compositions include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols, such as mannitol, sorbitol, or sodium chloride in the composition.

A pharmaceutical composition also may include a pharmaceutically acceptable anti-oxidant. These compositions may also contain adjuvants, such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents, which delay absorption such as aluminium monostearate and gelatin.

Therapeutic or diagnostic compositions typically must be sterile and stable under the conditions of manufacture and storage. The pharmaceutical composition can be formulated as a solution, microemulsion, liposome, or other ordered structure suitable to high drug concentration.

In further embodiments, the pharmaceutical compositions described herein can be administered by a route such as intravenous, subcutaneous, intraocular, intramuscular, intra-articular, intradermal, intraperitoneal, spinal or by other parenteral routes of administration, for example by injection or infusion. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a particularly preferred embodiment, the pharmaceutical composition is administered intravenously or subcutaneously. Hence, in one particularly preferred embodiment, the route of administration is intravenous. In another particularly preferred embodiment, the route of administration is subcutaneous. Subcutaneous injection is an especially preferred route. Intravenous administration by infusion or injection is also especially preferred as an administration route.

In one preferred embodiment, the antibody or fragment is provided in a pharmaceutical composition specifically formulated for injection. In one preferred embodiment, the antibody or fragment is provided in a pharmaceutical composition specifically formulated for subcutaneous injection. In one preferred embodiment, the antibody or fragment is provided in a pharmaceutical composition specifically formulated intravenous injection.

Sterile injectable solutions can be prepared by incorporating the active agent (e.g. antibody) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

The pharmaceutical composition employed may be presented in a preloaded device, for example it may be present in a preloaded syringe. In one embodiment, the pharmaceutical composition is in the form of an i.v. bag comprising the pharmaceutical composition. In one embodiment, the pharmaceutical composition is present in a vial.

Dosage unit form as used herein refers to physically discrete units suited as unitary dosages for the subjects to be treated; each unit contains a predetermined quantity of active compound calculated to produce the desired therapeutic effect in association with the required pharmaceutical carrier. In one preferred embodiment a preloaded device, i.v. bag, or vial is in unit dosage form.

Pharmaceutical compositions may comprise additional active ingredients as well as an antibody. As mentioned above, compositions may comprise one or more antibodies of the invention. They may also comprise additional active agents. Such additional agents may be conjugated to the antibody.

Also within the scope of the present invention are kits comprising antibodies or other compositions of the invention and instructions for use. The kit may further contain one or more additional reagents, such as an additional therapeutic or prophylactic agent as discussed herein.

The present invention is further illustrated by the following examples which should not be construed as further limiting. The contents of all figures and all references, patents and published patent applications cited throughout this application are expressly incorporated herein by reference.

### EXAMPLES

### Materials and Methods

### Collagen-Induced Arthritis

Male DBA/1JRj mice were purchased from Janvier-Elevage at 10-12 weeks of age. Mice were housed in conventional cages on a 12 h day-night cycle and fed standard chow ad libitum. To induce arthritis, bovine collagen II was diluted to a concentration of 2 mg/ml in 50 mM acetic acid and emulsified in equal volumes of Freund's complete adjuvant. On day 0, mice were immunized intradermally at the tail base with 100 μg bovine collagen II. On day 21, the mice received an intraperitoneal booster injection with 50 μg bovine CII dissolved in PBS. The onset of arthritis becomes visible a few days later.

Development of arthritis was scored 3 times a week, according to a 2-point scale for swelling and redness in each paw. Mice with an arthritis score of ≥1 with at least 1 paw having a score of ≥1 were included in the study.

Study protocols conformed to the Institute of Laboratory Animal Research guide for Laboratory Animals and were approved by the Radboudumc animal ethics committee.

### Conjugation and Radiolabeling

The antibody m-ACHA (or mACHA), which has equivalent target binding, NET inhibitory, and in vivo therapeutic properties to the CIT-013 antibody, was used in the experiments described herein. The mACHA antibody has the same CDRs as the CIT-013 antibody. In some of the Figures described herein therefore, the mACHA antibody may be referred to as CIT-013. mACHA and the mouse IgG1k isotype control (cIgG), anti-hen egg lysozyme, were conjugated with isothiocyanatobenzyl-diethylenetriaminepentaacetic acid (ITC-DTPA, Macrocyclics) in NaHCO3 (pH 9.5) for 1 h at room temperature at a molar excess of 10 ITC-DTPA molecules per antibody. The conjugated antibodies were dialyzed against PBS supplemented with Chelex 100 (Biorad) to remove the unbound chelator. Radiolabeling with indium-111-chloride (Curium) was performed using a 2-fold volume excess of the Indium-111-chloride of 2-(N-morpholino)ethanesulfonic acid (0.5 M MES, pH 5.5) buffer for 20 min at RT. Radiolabeling efficiency was >95% for all labeling procedures, as determined by instant thin-layer chromatography in 0.1 M sodium citrate buffer (Sigma Aldrich).

### Pharmacokinetics and Dose Optimization Studies

For in vivo studies the conjugated antibodies were labeled with indium-111 at a specific activity of 3.3 MBq per μg. Radiolabeled antibody was supplemented with unlabeled antibody to 3, 10 or 30 μg, with a final activity dose of 0.5 MBq for biodistribution and 10 MBq for SPECT/CT analysis as described in the sections below.

To study the pharmacokinetics and thereby determine the optimal imaging timepoint 30 healthy DBA/1JRj mice received an intravenous injection at the tail base of 10 μg [¹¹¹In]In-DTPA-mACHA. At 16, 24 or 48 hours post injection ten mice were sacrificed for biodistribution and SPECT/CT analysis. The optimal timepoint for imaging was determined to be 24 hours post injection.

Dose optimization studies were performed in the collagen induced arthritis (CIA) mouse model. CIA was induced in 40 mice as described above. Mice received 3, 10 or 30 μg of [111In]In-DTPA-mACHA, prepared as described above, n=10 per group. Mice were sacrificed after 24 hours for SPECT/CT and/or biodistribution. After determining the optimal dose, 10 additional mice received 10 μg of [¹¹¹In]In-DTPA-cIgG to determine uptake specificity.

### SPECT/CT

Mice for SPECT analysis were sacrificed and scanned for 60 minutes postmortem using a 1-mm-diameter pinhole ultra-high sensitivity mouse collimator (U-SPECT/CT-VI, MILabs). SPECT scans were followed by CT scans (65 kV, 615 μA). The SPECT/CT scans were reconstructed using VivoQuant™ software. Subsequently, biodistribution was performed.

### Biodistribution

Both the mice which received a biodistribution dose of 111In (n=8 per group) and SPECT mice (n=2) were sacrificed, and relevant tissues were dissected and weighed. Tissue uptake of 111In was determined using a T-counter (WIZARD, 2480 Automatic Gamma Counter, Perkin Elmer). Results are depicted as percentage of the injected amount per gram tissue (% IA/g).

### Statistical Analysis

The results are reported as the mean±standard deviation. All statistical analyses were performed using GraphPad Prism software version 10. Statistical tests used are mentioned in the figure legends. Results were considered significant at p<0.05.

### Example 1—Uptake of [¹¹¹In]In-mACHA in Tissues of Mice with Collagen Induced Arthritis

Antibody joint-to-blood ratio, average from front and hind paws, showed a ±100-fold increase of [¹¹¹In]In-DTPA-mACHA uptake in paws (FIG. 1). The higher [¹¹¹In]In-DTPA-cIgG blood level results in a blood-to-joint ratio of <1, indicate a specific uptake (n=10 per group resulting in 40 paws per group). The joint-to-blood ratio was significantly higher in the mACHA dosed groups compared to cIgG (****=p<0.0001), but not between mACHA dosed groups (Kruskal-Wallis test, Dunn's multiple comparisons).

### Example 2—Uptake of [¹¹¹In]in-DTPA-mACHA Correlates with Arthritic Score

Correlation of antibody uptake with increasing arthritic score was visualized for the front paws, for the four different dose groups, by using simple linear regression (FIG. 2). Uptake of [¹¹¹In]In-DTPA-mACHA was significantly increased (p=0.005) in mild arthritic paws (arthritic score=0.25, n=6) compared to unaffected paws (arthritic score=0, n=25), as calculated by Mann-Whitney test. Paws of all 3 dose groups were included in this calculation. Localization in mild arthritic paws is an indication of the unexpected sensitivity of the antibody.

### Example 3—SPECT/CT Images Showing Antibody Homing in Arthritic Joints

SPECT/CT imaging showed the specific localization of [¹¹¹In]In-DTPA-mACHA into the ankle and toes of the left hind paw, matching with the arthritic score of 1.25 (FIG. 3). Additional uptake could be seen in the left and right knee and left shoulder. These joints were not part of the scoring system as macroscopical swelling cannot be observed in those areas.

The Examples show that the antibodies or binding fragments described herein exhibit unexpected accuracy and sensitivity when used as diagnostic agents in an animal model of arthritis.

Sequences

SEQ ID NO: 1-CDR1 of msVH22.101 and hVH22.101(HC)x
GYTFTNYG
SEQ ID NO: 2-CDR2 of msVH22.101 and hVH22.101(HC)x
INTYSGEA
SEQ ID NO: 3-CDR3 of msVH22.101 and hVH22.101(HC)x
LRGYTYQSFDEGGDY
SEQ ID NO: 4-CDR2 of msVL22.101 and hVL22.101(LC)y
LVS
SEQ ID NO: 5-CDR3 of msVL22.101 and hVL22.101(LC)y
WQGTHFPYT
SEQ ID NO: 6-CDR1 of hVL22.101LC17
QSLLDTDGKTY
SEQ ID NO: 7-CDR1 of hVL22.101LC21
QSLLDSDAKTY
SEQ ID NO: 8-CDR1 of hVL22.101LC27
QSLLDTDAKTY
SEQ ID NO: 9-CDR1 of hVL22.101LC41
QSLLDADGKTY
SEQ ID NO: 10-CDR1 of hVL22.101LC42
QSLLDNDGKTY
SEQ ID NO: 11-hVH22.101f
RIQLVQSGAEVKKPGASVKVSCKASGYTFTNYGMHWVRQAPGQGLEWMGWINTYSGEATYAQKFQGRVTMTRDTSISTAYMELSRL
RSDDTAVYYCLRGYTYQSFDEGGDYWGQGTLVTVSS
SEQ ID NO: 12-hVH22.101HC9
RIQLVQSGAEVKKPGASVKVSCKASGYTFTNYGMHWVRQAPGQGLEWMGWINTYSGEATYVDDFQGRVTMTRDTSISTAYMELSRL
RSDDTAVYYCLRGYTYQSFDEGGDYWGQGTLVTVSS
SEQ ID NO: 13-hVL22.101LC17
DVVMTQSPLSLPVTLGQPASISCRSSQSLLDTDGKTYLNWFQQRPGQSPRRLIYLVSKLDSGVPDRFSGSGSGTDFTLKISRVEAE
DVGVYYCWQGTHFPYTFGQGTKLEIK
SEQ ID NO: 14-hVL22.101LC21
DVVMTQSPLSLPVTLGQPASISCRSSQSLLDSDAKTYLNWFQQRPGQSPRRLIYLVSKLDSGVPDRFSGSGSGTDFTLKISRVEAE
DVGVYYCWQGTHEPYTFGQGTKLEIK
SEQ ID NO: 15-hVL22.101LC27
DVVMTQSPLSLPVTLGQPASISCRSSQSLLDTDAKTYLNWFQQRPGQSPRRLIYLVSKLDSGVPDRFSGSGSGTDFTLKISRVEAE
DVGVYYCWQGTHFPYTFGQGTKLEIK
SEQ ID NO: 16-hVL22.101LC41
DVVMTQSPLSLPVTLGQPASISCRSSQSLLDADGKTYLNWFQQRPGQSPRRLIYLVSKLDSGVPDRFSGSGSGTDFTLKISRVEAE
DVGVYYCWQGTHFPYTFGQGTKLEIK
SEQ ID NO: 17-hVL22.101LC42
DVVMTQSPLSLPVTLGQPASISCRSSQSLLDNDGKTYLNWFQQRPGQSPRRLIYLVSKLDSGVPDRFSGSGSGTDFTLKISRVEAE
DVGVYYCWQGTHEPYTFGQGTKLEIK
SEQ ID NO: 18-SEQ ID NO 1 from WO2016092082 (used in Example 1/7) from histone 2A
SGXGKQGGKARA
Where X is citrulline
SEQ ID NO: 19-SEQ ID NO 2 from WO2016092082, (used in Example 7) from histone 4
SGXGKGGKGLGKGGAKRHRKVLR
Where X is citrulline
SEQ ID NO: 20-Shortened SEQ ID NO 2 from WO2016092082 (used in Example 7) from histone 4
SGXGKGGKGLGK
Where X is citrulline
SEQ ID NO: 21-Peptide no 4 (human histone 2A) (SEQ ID NO 24 from WO2011070172)
QFPVGXVHRLLR
Where X is citrulline
SEQ ID NO: 22-Peptide no 6 (human histone 2A) (SEQ ID NO 26 from WO2011070172)
VHRLLXKGNYSE
Where X is citrulline
SEQ ID NO: 23-Human heavy chain constant domain of IgG1
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPG
SEQ ID NO: 24-Human kappa chain constant domain
RTVAAPSVFIFPPSDEQLKSGTASVVCLLNNFYPREAKVQWKVDNALQSGNSQESVTEQDSKDSTYSLSSTLTLSKADYEKHKVYA
CEVTHQGLSSPVTKSFNRGEC
SEQ ID NO: 25-ms VH22.101
RIQLVQSGPELKKPGEAVKISCKASGYTFTNYGMHWMKQTPGKDFRWMGWINTYSGEATYVDDFKGRFAFSLGTSASTAYLQINNL
KNDDTATYFCLRGYTYQSFDEGGDYWGQGTALTVSS
SEQ ID NO: 26-hVH22.101j
QVQLVQSGAEVKKPGASVKVSCKASGYTFTNYGMHWVRQAPGQGLEWMGWINTYSGEATYAQKFQGRVTMTRDTSISTAYMELSRL
RSDDTAVYYCLRGYTYQSFDEGGDYWGQGTLVTVSS
SEQ ID NO: 27-hVH22.101HC7
QVQLVQSGAEVKKPGSSVKVSCKASGYTFTNYGMHWVRQAPGQGLEWMGWINTYSGEATYAQKFQGRVTITADESTSTAYMELSSL
RSEDTAVYYCLRGYTYQSFDEGGDYWGQGTLVTVSS
SEQ ID NO: 28-hVH22.101HC8
QVQLVQSGAEVKKPGSSVKVSCKASGYTFTNYGMHWVRQAPGQGLEWMGWINTYSGEATYVDDFQGRVTITADESTSTAYMELSSL
RSEDTAVYYCLRGYTYQSFDEGGDYWGQGTLVTVSS
SEQ ID NO: 29-hVH22.101HC10
QVQLVQSGAEVKKPGASVKVSCKASGYTFTNYGMHWVRQAPGQGLEWMGWINTYSGEATYVDDFQGRVTMTRDTSISTAYMELSRL
RSDDTAVYYCLRGYTYQSFDEGGDYWGQGTLVTVSS
SEQ ID NO: 30-msVL22.101
DVVMTQTPLTLSVTTGQPASISCKSSQSLLDSDGKTYLNWLFQRPGQSPKRLIYLVSKLDSGVPDRFTGSGSGTDFTLKISRVEAE
DLGI YYCWQGTHFPYTFGGGTNLEIK
SEQ ID NO: 31-hVL22.101e
DVVMTQSPLSLPVTLGQPASISCRSSQSLVDSDGKTYLNWFQQRPGQSPRRLIYLVSKLDSGVPDRFSGSGSGTDFTLKISRVEAE
DVGVYYCWQGTHFPYTFGQGTKLEIK
SEQ ID NO: 32-hVL22.101g
DVVMTQSPLSLPVTLGQPASISCRSSQSLLDSDGKTYLNWFQQRPGQSPRRLIYLVSKLDSGVPDRFSGSGSGTDFTLKISRVEAE
DVGVYYCWQGTHFPYTFGQGTKLEIK
SEQ ID NO: 33-hVL22.101h
DVVMTQSPLSLPVTLGQPASISCRSSQSLVASDGKTYLNWFQQRPGQSPRRLIYLVSKLDSGVPDRFSGSGSGTDFTLKISRVEAE
DVGVYYCWQGTHEPYTFGQGTKLEIK
SEQ ID NO: 34-hVL22.101i
DVVMTQSPLSLPVTLGQPASISCRSSQSLVESDGKTYLNWFQQRPGQSPRRLIYLVSKLDSGVPDRFSGSGSGTDFTLKISRVEAE
DVGVYYCWQGTHFPYTFGQGTKLEIK
SEQ ID NO: 35-hVL22.101j
DVVMTQSPLSLPVTLGQPASISCRSSQSLVSSDGKTYLNWFQQRPGQSPRRLIYLVSKLDSGVPDRFSGSGSGTDFTLKISRVEAE
DVGVYYCWQGTHEPYTFGQGTKLEIK
SEQ ID NO: 36-CDR1 of msVL22.101 and hVL22.101g
QSLLDSDGKTY
SEQ ID NO: 37-CDR1 of hVL22.101e
QSLVDSDGKTY
SEQ ID NO: 38-CDR1 of hVL22.101h
QSLVASDGKTY
SEQ ID NO: 39-CDR1 of hVL22.101i
QSLVESDGKTY
SEQ ID NO: 40-CDR1 of hVL22.101j
QSLVSSDGKTY
SEQ ID NO: 41-CDR1 of hVL22.101LC16
QSLLESDGKTY
SEQ ID NO: 42-CDR1 of hVL22.101LC19
QSLLDSEGKTY
SEQ ID NO: 43-CDR1 of hVL22.101LC20
QSLLDSSGKTY
SEQ ID NO: 44-CDR1 of hVL22.101LC22
QSLLESEGKTY
SEQ ID NO: 45-CDR1 of hVL22.101LC23
QSLLESSGKTY
SEQ ID NO: 46-CDR1 of hVL22.101LC24
QSLLESDAKTY
SEQ ID NO: 47-CDR1 of hVL22.101LC25
QSLLDTEGKTY
SEQ ID NO: 48-CDR1 of hVL22.101LC26
QSLLDTSGKTY
SEQ ID NO: 49-CDR1 of hVL22.101LC37
QSLLDSAGKTY
SEQ ID NO: 50-CDR1 of hVL22.101LC38
QSLLESAGKTY
SEQ ID NO: 51-CDR1 of hVL22.101LC39
QSLLDAEGKTY
SEQ ID NO: 52-CDR1 of hVL22.101LC40
QSLLDNEGKTY
SEQ ID NO: 53-msFibß XG (SEQ ID NO 37 from WO2011070172)
EPTDSLDAXGHRPVDRR
Where X is citrulline
SEQ ID NO: 54-ms Vim XS/XL (SEQ ID NO 38 from WO2011070172)
YVTXSSAVXLXSSVP
Where X is citrulline
SEQ ID NO: 55-Region around CDR2 of msVL22.101 and hVL22.101(LC)y
LVSKLDS
SEQ ID NO: 56-Heavy chain constant domain of hCH22.101f
ASTKGPSVFPLAPSSKSTSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK

What is claimed is:

1. A method of identifying or diagnosing a human subject with a pathology associated with extracellular trap formation or release from cells comprising the use of an antibody or antigen binding fragment thereof that specifically binds to a citrullinated epitope on deiminated human histone 2A and/or histone 4 as a diagnostic agent.

2. The method of claim 1, wherein the antibody or binding fragment thereof is conjugated to an additional moiety.

3. The method of claim 2, wherein the antibody or antigen binding fragment thereof is labelled.

4. The method of claim 3, wherein diagnosing said pathology is dependent on the binding of the antibody or binding fragment thereof to a citrullinated epitope on deiminated human histone 2A and/or histone 4.

5. A method of identifying or diagnosing a human subject with a pathology associated with extracellular trap formation or release from cells comprising administering a labelled antibody or antigen binding fragment thereof that specifically binds to a citrullinated epitope on deiminated human histone 2A and/or histone 4 to said subject, and diagnosing said pathology based on the localisation of the labelled antibody or antigen binding fragment in the human subject.

6. A method of identifying or diagnosing a human subject with a pathology associated with extracellular trap formation or release from cells comprising acquiring an image of the human subject to whom an amount of a labelled antibody or antigen binding fragment thereof that specifically binds to a citrullinated epitope on deiminated human histone 2A and/or histone 4 has been administered, wherein diagnosing said pathology is based on the localisation of the labelled antibody or antigen binding fragment in the image.

7. The method of any one of claims 3 to 6, wherein the label is a fluorescent, an enzymatic, bioluminescent or a radioactive label.

8. The method of claim 7, wherein the radioactive label is 67Cu, 99mTc, 111In, 123I, 131I, 155Tb, 186Rh, 188Rh, 11C, 18F, 43/44Sc, 52Mn, 64/66Cu, 86Y, 89Zr, 124I or 152Tb.

9. The method according to any one of the preceding claims, wherein the pathology associated with extracellular trap formation or release from cells is a Macrophage Extracellular Trap (MET)-associated pathology, a Mast Cell Extracellular Trap (MCET)-associated pathology, a Monocyte derived Extracellular Trap-associated pathology, a NET-associated pathology or an EET-associated pathology.

10. The method according to claim 9, wherein the NET-associated pathology is an inflammatory condition, an autoimmune condition or another NET-associated pathology such as wound healing in diabetes, cancer, cancer metastasis, transplant organ health in vivo or ex vivo, heart failure or viral induced acute respiratory distress syndrome (ARDS).

11. The method according to claim 10, wherein the condition is systemic lupus erythematosus (SLE), idiopathic inflammatory myopathy, lupus, sepsis, thrombosis, vasculitis, small-vessel vasculitis (SVV), antiphospholipid syndrome (APS), inflammatory arthritis, rheumatoid arthritis and osteoarthritis, psoriasis, dermatomyositis, polymyositis, inclusion body myositis, hidradenitis suppurativa, Alzheimer's disease, autoimmune hepatitis, juvenile idiopathic arthritis, ulcerative colitis, myositis (polymyositis and dermatomyositis), Sjögren's disease, Anti-phospholipid Syndrome, Bechet's disease, spondylitis, spondyloarthropathy, multiple system atrophy, Parkinson's disease, Lewy body dementia asthma, heart failure with preserved ejection fraction, heart failure with reduced ejection fraction, asthma, allergic rhinovirus exacerbated asthma, allergic asthma, cystic fibrosis, fibrosis and idiopathic pulmonary fibrosis, idiopathic pulmonary fibrosis, atherosclerosis, dry eye disease, uveitis, nongranulomatous uveitis, granulomatous uveitis, dermatitis, atopic dermatitis, inflammatory bowel disease, COPD or bronchitis.

12. The method according to claim 11, wherein the condition is hidradenitis suppurativa, inflammatory arthritis, rheumatoid arthritis or osteoarthritis.

13. The method according to any one of claims 10 to 12, wherein NET-associated pathology or condition is diagnosed at an early stage.

14. The method according to claim 9, wherein the EET-associated pathology is an eosinophilic disease or condition is an eosinophilic disease or condition of the skin; a respiratory eosinophilic disease or condition; a gastro-intestinal eosinophilic disease or condition; an allergic disease or condition; or a helminth, fungal, viral, or bacterial infection.

15. The method according to claim 14, wherein said eosinophilic disease or condition is an eosinophilic disease or condition of the skin; a respiratory eosinophilic disease or condition; a gastro-intestinal eosinophilic disease or condition; an allergic disease or condition; or a helminth, fungal, viral, or bacterial infection.

16. The method according to claim 14 or 15, wherein the EET-associated pathology or eosinophilic disease or condition is selected from: Bullous Pemphigoid, Atopic dermatitis, allergic contact dermatitis, Eosinophilic Asthma, Chronic RhinoSinusitis with Nasal Polyposis (CRSwNP), Allergic sinusitis, Allergic bronchopulmonary aspergillosis, Eosinophilic chronic rhinosinusitis, Eosinophilic Esophagitis (EoE), HyperEosinophilic Syndrome (HES), Eosinophilic Granulomatosis with PolyAngitis (EGPA), Eosinophilic otitis media (EOM), and Drug Reaction with Eosinophilic & Systemic Symptoms (DRESS), arteriosclerosis, and vasculitis.

17. The method according to any one of claims 14 to 16, wherein the EET-associated pathology or eosinophilic disease or condition is selected from: Eosinophilic Asthma, Chronic RhinoSinusitis with Nasal Polyposis (CRSwNP), Eosinophilic chronic rhinosinusitis, Eosinophilic otitis media (EOM), arteriosclerosis and vasculitis.

18. The method according to any one of the preceding claims, wherein the antibody or binding fragment thereof comprises the CDRs of SEQ ID NOs: 1, 2, 3, 9, 4 and 5.

19. The method according to claim 18, wherein the antibody or binding fragment thereof comprises:
a) the heavy chain variable domain amino acid sequence of SEQ ID NO: 11 and the light chain variable domain amino acid sequence of SEQ ID NO: 13;
b) the heavy chain variable domain amino acid sequence of SEQ ID NO: 11 and the light chain variable domain amino acid sequence of SEQ ID NO: 14;
c) the heavy chain variable domain amino acid sequence of SEQ ID NO: 11 and the light chain variable domain amino acid sequence of SEQ ID NO: 15;
d) the heavy chain variable domain amino acid sequence of SEQ ID NO: 11 and the light chain variable domain amino acid sequence of SEQ ID NO: 16;
e) the heavy chain variable domain amino acid sequence of SEQ ID NO: 11 and the light chain variable domain amino acid sequence of SEQ ID NO: 17;
f) the heavy chain variable domain amino acid sequence of SEQ ID NO: 12 and the light chain variable domain amino acid sequence of SEQ ID NO: 13;
g) the heavy chain variable domain amino acid sequence of SEQ ID NO: 12 and the light chain variable domain amino acid sequence of SEQ ID NO: 14;
h) the heavy chain variable domain amino acid sequence of SEQ ID NO: 12 and the light chain variable domain amino acid sequence of SEQ ID NO: 15;
i) the heavy chain variable domain amino acid sequence of SEQ ID NO: 12 and the light chain variable domain amino acid sequence of SEQ ID NO: 16; or
j) the heavy chain variable domain amino acid sequence of SEQ ID NO: 12 and the light chain variable domain amino acid sequence of SEQ ID NO: 17.

20. The method according to any one of the preceding claims, wherein the antibody or binding fragment thereof is selected from the group consisting of recombinant antibodies, single chain antibodies, single chain variable fragments (scFv), variable fragments (Fv), fragment antigen-binding regions (Fab), single-domain antibodies (sdAb), VHH antibodies, nanobodies, camelids-derived single-domain antibodies, shark IgNAR-derived single-domain antibody fragments (VNAR), diabodies, triabodies, Anticalins and aptamers.

21. The method according to any one of claims 1 to 19, wherein the antibody is a full-length antibody.

22. The method according to any one of the preceding claims, wherein the antibody or binding fragment thereof comprises an Fc region, such as an IgG1, IgG2, IgG3 or IgG4 region.

23. The method according to any one of claims 18 to 22, wherein the heavy chain constant region comprises SEQ ID NO: 23 or 56, and/or the light chain constant region comprises SEQ ID NO: 24, optionally wherein the antibody comprises the heavy chain variable domain amino acid sequence of SEQ ID NO: 11, the light chain variable domain amino acid sequence of SEQ ID NO: 16, the heavy chain constant region amino acid sequence of SEQ ID NO: 23 or 56, and the light chain constant region amino acid sequence of SEQ ID NO: 24.

24. The method of any one of the preceding claims, wherein the human subject identified or diagnosed with a pathology associated with extracellular trap formation or release from cells is subsequently treated with an agent that inhibits the formation of extracellular traps.

25. The method of claim 24, wherein the agent is an antibody or binding fragment thereof as defined in any one of claims 18 to 23.


SEQUENCE LISTING (56 sequences)

SEQ ID NO: 1 - protein, 8 aa, synthetic construct
  REGION 1..8: note = CDR1 of msVH22.101 and hVH22.101(HC)x
GYTFTNYG

SEQ ID NO: 2 - protein, 8 aa, synthetic construct
  REGION 1..8: note = CDR2 of msVH22.101 and hVH22.101(HC)x
INTYSGEA

SEQ ID NO: 3 - protein, 15 aa, synthetic construct
  REGION 1..15: note = CDR3 of msVH22.101 and hVH22.101(HC)x
LRGYTYQSFDEGGDY

SEQ ID NO: 4 - skipped

SEQ ID NO: 5 - protein, 9 aa, synthetic construct
  REGION 1..9: note = CDR3 of msVL22.101 and hVL22.101(LC)y
WQGTHFPYT

SEQ ID NO: 6 - protein, 11 aa, Homo sapiens
QSLLDTDGKTY

SEQ ID NO: 7 - protein, 11 aa, Homo sapiens
QSLLDSDAKTY

SEQ ID NO: 8 - protein, 11 aa, Homo sapiens
QSLLDTDAKTY

SEQ ID NO: 9 - protein, 11 aa, Homo sapiens
QSLLDADGKTY

SEQ ID NO: 10 - protein, 11 aa, Homo sapiens
QSLLDNDGKTY

SEQ ID NO: 11 - protein, 122 aa, synthetic construct
  REGION 1..122: note = hVH22.101f
RIQLVQSGAEVKKPGASVKVSCKASGYTFTNYGMHWVRQAPGQGLEWMGWINTYSGEATYAQKFQGRVTMTRDTSISTAYMELSRLRSDDTAVYYCLRGYTYQSFDEGGDYWGQGTLVTVSS

SEQ ID NO: 12 - protein, 122 aa, Homo sapiens
RIQLVQSGAEVKKPGASVKVSCKASGYTFTNYGMHWVRQAPGQGLEWMGWINTYSGEATYVDDFQGRVTMTRDTSISTAYMELSRLRSDDTAVYYCLRGYTYQSFDEGGDYWGQGTLVTVSS

SEQ ID NO: 13 - protein, 112 aa, Homo sapiens
DVVMTQSPLSLPVTLGQPASISCRSSQSLLDTDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 14 - protein, 112 aa, Homo sapiens
DVVMTQSPLSLPVTLGQPASISCRSSQSLLDSDAKTYLNWFQQRPGQSPRRLIYLVSKLDSGVPDRFSGSGSGTDFTLKISRVEAEDVGVYYCWQGTHFPYTFGQGTKLEIK

SEQ ID NO: 15 - protein, 112 aa, Homo sapiens
DVVMTQSPLSLPVTLGQPASISCRSSQSLLDTDAKTYLNWFQQRPGQSPRRLIYLVSKLDSGVPDRFSGSGSGTDFTLKISRVEAEDVGVYYCWQGTHFPYTFGQGTKLEIK

SEQ ID NO: 16 - protein, 112 aa, Homo sapiens
DVVMTQSPLSLPVTLGQPASISCRSSQSLLDADGKTYLNWFQQRPGQSPRRLIYLVSKLDSGVPDRFSGSGSGTDFTLKISRVEAEDVGVYYCWQGTHFPYTFGQGTKLEIK

SEQ ID NO: 17 - protein, 112 aa, Homo sapiens
DVVMTQSPLSLPVTLGQPASISCRSSQSLLDNDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 18 - protein, 12 aa, Homo sapiens
  SITE 3: note = X = citrulline
SGXGKQGGKARA

SEQ ID NO: 19 - protein, 23 aa, Homo sapiens
  SITE 3: note = X = citrulline
SGXGKGGKGLGKGGAKRHRKVLR

SEQ ID NO: 20 - protein, 12 aa, Homo sapiens
  SITE 3: note = X = citrulline
SGXGKGGKGLGK

SEQ ID NO: 21 - protein, 12 aa, Homo sapiens
  SITE 6: note = X = citrulline
QFPVGXVHRLLR

SEQ ID NO: 22 - protein, 12 aa, Homo sapiens
  SITE 6: note = X = citrulline
VHRLLXKGNYSE

SEQ ID NO: 23 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 107 aa, Homo sapiens [sequence omitted]

SEQ ID NO: 25 - protein, 122 aa, Mus musculus
RIQLVQSGPELKKPGEAVKISCKASGYTFTNYGMHWMKQTPGKDFRWMGWINTYSGEATYVDDFKGRFAFSLGTSASTAYLQINNLKNDDTATYFCLRGYTYQSFDEGGDYWGQGTALTVSS

SEQ ID NO: 26 - protein, 122 aa, Homo sapiens
QVQLVQSGAEVKKPGASVKVSCKASGYTFTNYGMHWVRQAPGQGLEWMGWINTYSGEATYAQKFQGRVTMTRDTSISTAYMELSRLRSDDTAVYYCLRGYTYQSFDEGGDYWGQGTLVTVSS

SEQ ID NO: 27 - protein, 122 aa, Homo sapiens
QVQLVQSGAEVKKPGSSVKVSCKASGYTFTNYGMHWVRQAPGQGLEWMGWINTYSGEATYAQKFQGRVTITADESTSTAYMELSSLRSEDTAVYYCLRGYTYQSFDEGGDYWGQGTLVTVSS

SEQ ID NO: 28 - protein, 122 aa, Homo sapiens
QVQLVQSGAEVKKPGSSVKVSCKASGYTFTNYGMHWVRQAPGQGLEWMGWINTYSGEATYVDDFQGRVTITADESTSTAYMELSSLRSEDTAVYYCLRGYTYQSFDEGGDYWGQGTLVTVSS

SEQ ID NO: 29 - protein, 122 aa, Homo sapiens
QVQLVQSGAEVKKPGASVKVSCKASGYTFTNYGMHWVRQAPGQGLEWMGWINTYSGEATYVDDFQGRVTMTRDTSISTAYMELSRLRSDDTAVYYCLRGYTYQSFDEGGDYWGQGTLVTVSS

SEQ ID NO: 30 - protein, 112 aa, Mus musculus
DVVMTQTPLTLSVTTGQPASISCKSSQSLLDSDGKTYLNWLFQRPGQSPKRLIYLVSKLDSGVPDRFTGSGSGTDFTLKISRVEAEDLGIYYCWQGTHFPYTFGGGTNLEIK

SEQ ID NO: 31 - protein, 112 aa, Homo sapiens
DVVMTQSPLSLPVTLGQPASISCRSSQSLVDSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 32 - protein, 112 aa, Homo sapiens
DVVMTQSPLSLPVTLGQPASISCRSSQSLLDSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 33 - protein, 112 aa, Homo sapiens
DVVMTQSPLSLPVTLGQPASISCRSSQSLVASDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 34 - protein, 112 aa, Homo sapiens
DVVMTQSPLSLPVTLGQPASISCRSSQSLVESDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 35 - protein, 112 aa, Homo sapiens
DVVMTQSPLSLPVTLGQPASISCRSSQSLVSSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 36 - protein, 11 aa, synthetic construct
  REGION 1..11: note = CDR1 of msVL22.101 and hVL22.101g
QSLLDSDGKTY

SEQ ID NO: 37 - protein, 11 aa, Homo sapiens
QSLVDSDGKTY

SEQ ID NO: 38 - protein, 11 aa, Homo sapiens
QSLVASDGKTY

SEQ ID NO: 39 - protein, 11 aa, Homo sapiens
QSLVESDGKTY

SEQ ID NO: 40 - protein, 11 aa, Homo sapiens
QSLVSSDGKTY

SEQ ID NO: 41 - protein, 11 aa, Homo sapiens
QSLLESDGKTY

SEQ ID NO: 42 - protein, 11 aa, Homo sapiens
QSLLDSEGKTY

SEQ ID NO: 43 - protein, 11 aa, Homo sapiens
QSLLDSSGKTY

SEQ ID NO: 44 - protein, 11 aa, Homo sapiens
QSLLESEGKTY

SEQ ID NO: 45 - protein, 11 aa, Homo sapiens
QSLLESSGKTY

SEQ ID NO: 46 - protein, 11 aa, Homo sapiens
QSLLESDAKTY

SEQ ID NO: 47 - protein, 11 aa, Homo sapiens
QSLLDTEGKTY

SEQ ID NO: 48 - protein, 11 aa, Homo sapiens
QSLLDTSGKTY

SEQ ID NO: 49 - protein, 11 aa, Homo sapiens
QSLLDSAGKTY

SEQ ID NO: 50 - protein, 11 aa, Homo sapiens
QSLLESAGKTY

SEQ ID NO: 51 - protein, 11 aa, Homo sapiens
QSLLDAEGKTY

SEQ ID NO: 52 - protein, 11 aa, Homo sapiens
QSLLDNEGKTY

SEQ ID NO: 53 - protein, 17 aa, Homo sapiens
  SITE 9: note = X = citrulline
EPTDSLDAXGHRPVDRR

SEQ ID NO: 54 - protein, 15 aa, Homo sapiens
  SITE 4: note = X = citrulline
  SITE 9: note = X = citrulline
  SITE 11: note = X = citrulline
YVTXSSAVXLXSSVP

SEQ ID NO: 55 - protein, 7 aa, synthetic construct
  REGION 1..7: note = Region around CDR2 of msVL22.101 and hVL22.101(LC)y
LVSKLDS

SEQ ID NO: 56 - protein, 330 aa, Homo sapiens [sequence omitted]